(19) United States
(12) Patent Application Publication
(10) Pub. No.: US 2024/0010971 A1
(43) Pub. Date: Jan. 11, 2024

(54) SPORE COMPOSITIONS, PRODUCTION AND USES THEREOF

(71) Applicant: BASF SE, Ludwigshafen am Rhein (DE)

(72) Inventors: Tobias MAY, Ludwigshafen (DE); Reinhard STIERL, Limburgerhof (DE); Hsuan CHANG, Bonn (DE); Daniel Christoph HEINRICH, Ludwigshafen (DE); Evangelina Priya HAAS, Ludwigshafen (DE); Andrea HEROLD, Ludwigshafen (DE)

(21) Appl. No.: 18/253,833

(22) PCT Filed: Dec. 10, 2021
(86) PCT No.: PCT/EP2021/085278

§ 371 Date: May 22, 2023

(30) Foreign Application Priority Data
Dec. 17, 2020 (EP) 20215121.3

(51) Int. Cl. C12N 1/20; A23L 33/135

(52) U.S. Cl. CPC C12N 1/20; A23L 33/135

(57) ABSTRACT

Disclosed herein are spore compositions and methods of producing such compositions. Additionally disclosed herein are plant protection products benefiting from such spore compositions and methods of using such compositions for the benefit of plants, for the reduction of pathogen emissions to nearby areas and for the benefit of animals or humans. Further disclosed herein are methods of efficient fermentation.

The present invention is concerned with providing spore compositions and methods of producing such compositions. The invention also is concerned with plant protection products and benefiting from such spore compositions and uses of such compositions for the benefit of plants, reduction of pathogen emissions to nearby areas and for the benefit of animals or humans. Furthermore, the invention is concerned with methods of efficient fermentation.

### BACKGROUND

The formation of endospores is a stage in the life cycle of several prokaryotic microorganisms. The main attribute of importance of endospores is that they provide a dormant life stage which typically provides resistance of the dormant cells against heat treatment (typically at least 5 minutes at 70° C., 1024 hPa) and other environmental conditions inimical to actively growing microorganisms like desiccation, ultraviolet radiation and chemical disinfectants. Sporulated microorganisms are thus capable of enduring long periods of harmful conditions. When conditions turn out to be favorable again, the sporulated cells germinate and turn into actively growing life stages. Endospores thus have a particular application for storage and fast retrieval of microorganisms. In particular endospores are used in agronomical and biotechnological products where easy product storage, long shelf life without elaborate storage conditions like liquid nitrogen and fast and reliable revival of microorganisms are required. In agronomical products, for example, microorganisms are desired that benefit plant health. In human nutrition and health care, application of probiotic organisms in the form of spores that can survive the low pH of the stomach enables targeted outgrowth in the gut to prevent digestive disorders. However, storage of such products should be independent of storage conditions and should preferably allow to store the product even at warm temperatures of 37-45° C. or, less preferred, exposed to sunlight. Upon application of the product the microorganisms should quickly and reliably multiply and exert their beneficial properties. In other products, viability of the microorganism is not required. However, endospores allow to compartmentalize and attach desired metabolites on its surface, for example biochemical pesticides. An advantage of such products is that they can avoid overuse of conventional pesticides and the drawbacks attached to such overuse, e.g. soil acidification. In biotechnological products reliable fermentations are usually required. Fermentation starts by inoculating a fermenter comprising a suitable growth medium with an aliquot of microorganisms, such that the microorganisms multiply during fermentation and produce the desired compounds as well as spores. To achieve reliable inoculation it is necessary to store, for extended periods, the inoculation aliquots such that they maintain their capacity to multiply and metabolize as required.

One major application of bacterial spores is its use in probiotic products for human and animal health, including the reduction and replacement of antibiotics. For instance, Clostridium species can utilize a broad variety of nutrients that cannot be digested by humans and animals. As an example, present in intestinal tract, Clostridia can convert indigestible polysaccharide to produce short-chain fatty acids (SCFAs), which can easily absorbed in the intestinal tract of the host and thus play a crucial role in intestinal homeostasis (Pingting Guo, Ke Zhang, Xi Ma and Pingli He, Clostridium species as probiotics: potentials and challenges, Journal of Animal Science and Biotechnology (2020) doi.org/10.1186/s40104-019-0402). SCFAs such as butyrate orchestrate multiple physiological functions to optimize luminal environment and maintain intestinal health. Several beneficial traits for health care applications using Clostridia are known, such as a crosstalk between Clostridium species and intestinal immune system inducing anti-inflammatory effects and improved gut immune tolerance. As an example, Clostridia were found out to attenuate colitis and allergic diarrhea of mice. Among with other species, some Clostridia are known to produce bile acid preventing cautious infections with toxigenic C. difficile. Application of protein or amino acid fermenting Clostridia can prevent excessive accumulation of ammonia that could directly and indirectly damage the intestinal epithelial cells. Beneficial traits of probiotic and prebiotic use of Clostridia were also known in dietary nutrition as well as in growth improvement in the livestock farming. Specific strains such as Clostridium estertheticum were applied as protective cultures in raw meat and poultry, fish and seafood products (Jones R, Zagorec M, Brightwell G, Tagg J R (2009) Inhibition by Lactobacillus sakei of other species in the flora of vacuum packaged raw meats during prolonged storage. Food microbiol 25:876-881).

In agriculture, bacterial spores were used in plant pest control compositions reducing or preventing phytopathogenic fungal or bacterial diseases. Spore biologicals are also applied to improve plants resistance against biotic and abiotic stress, to accelerate the growth of the plant and to increase the yield during plant, fruit or legume harvest. Spore products were applied to leaves, shoots, fruits, roots or plant propagation material as well as to the substrate where the plants are to grow (Toyota K. Bacillus-related Spore Formers: Attractive Agents for Plant Growth Promotion. Microbes Environ. 2015; 30(3):205-207. doi:10.1264/jsme2.me3003rh). Bochow, H., et al. “Use of Bacillus Subtilis as Biocontrol Agent. IV. Salt-Stress Tolerance Induction by Bacillus Subtilis FZB24 Seed Treatment in Tropical Vegetable Field Crops, and Its Mode of Action/Die Verwendung von Bacillus subtilis zur biologischen Bekämpfung. IV. Induktion einer Salzstress-Toleranz durch Applikation von Bacillus subtilis FZB24 bei tropischem Feldgemüse und sein Wirkungsmechanismus.” Zeitschrift für Pflanzenkrankheiten und Pflanzenschutz/Journal of Plant Diseases and Protection, vol. 108, no. 1, 2001, pp. 21-30. JSTOR, www.jstor.org/stable/43215378. Accessed 14 Dec. 2020.)(Hashem, Abeer & Tabassum, B. & Abd_Allah, Elsayed. (2019). Bacillus subtilis: A plant-growth promoting rhizobacterium that also impacts biotic stress. Saudi Journal of Biological Sciences. 26. 10.1016/j.sjbs.2019.05.004.)

Furthermore, bacterial spores were applied in the area of nanobiotechnology and building chemistry such as for self-healing concrete (crack healing), mortar stability and reduced water permeability [J. Y. Wang, H. Soens, W. Verstraete, N. De Belie, Self-healing concrete by use of microencapsulated bacterial spores, Cement and Concrete Research, Volume 56, 2014, 139-152, ISSN 0008-8846, https://doi.org/10.1016/j.cemconres.2013.11.009] [Ricca E, Cutting S M. Emerging Applications of Bacterial Spores in Nanobiotechnology. J Nanobiotechnology. 2003; 1(1):6. Published 2003 Dec. 15. doi:10.1186/1477-3155-1-6].

Additionally, bacterial spores were applied in the area of cleaning products, such as for cleaning of laundry, hard surfaces, sanitation and odor control (Caselli E. Hygiene: microbial strategies to reduce pathogens and drug resistance in clinical settings. Microb Biotechnol. 2017 September; 10(5):1079-1083. doi: 10.1111/1751-7915.12755. Epub 2017 Jul. 5) in the clinical and domestic setting. As an example, spores were used in cosmetic compositions such as skin cleaning products (US20070048244), for dishwashing agents (WO2014/107111), pipe degreasers (DE19850012), malodor control of laundry (WO2017/157778 and EP3430113) or the removal of allergens (US20020182184). Spores can also be embedment into non-biogenic matrixes to catalyze its subsequent breakdown.

Formation of endospores is thus an area of active research. However, mechanisms underlying sporulation differ between microorganisms. In Bacillus, Spo0A is phosphorylated (Spo0A_P) by a phosphorelay system initiated by orphan histidine kinases (HKs), in particular KinA and KinB. Subsequently, Spo0A_P initiates the sporulation sigma factor cascade involving four downstream sigma factors (σ^{F}, σ^{E}, σ^{G}, and σ^{K}). In contrast, no phosphorelay system is present in Clostridia which do directly transfer a phosphate group to Spo0A, thus activating it. As a consequence, preliminary sporulation stage 0 regulators such as Spo0B found in Bacilli and many Paenibacilli are not present in Clostridia.

Furthermore, the last sigma factor in the Bacillus model, 6K, was identified to play a double role in Clostridium, one early, upstream of Spo0A, and another late, downstream of GG, which is analogous to its role in Bacillus [Al-Hinai M A, Jones S W, Papoutsakis E T. The Clostridium sporulation programs: diversity and preservation of endospore differentiation. Microbiol Mol Biol Rev. 2015 March; 79(1):19-37. doi: 10.1128/MMBR.00025-14] [Tojo S, Hirooka K, Fujita Y. Expression of kinA and kinB of Bacillus subtilis, Necessary for Sporulation Initiation, Is under Positive Stringent Transcription Control, Journal of Bacteriology March 2013, 195 (8) 1656-1665; DOI: 10.1128/JB.02131-12].

On the other hand, as an example, entry into sporulation of B. subtilis is under control of RapA. This phosphatase is regulating the transcription of the master transcriptional regulator of all endospore formers, Spo0A and thus can act as a direct repressor [Perego M, Hanstein C, Welsh C. M., Djavakhishvili T., Glaser P., Hoch J. A. Multiple protein-aspartate phosphatases provide a mechanism for the integration of diverse signals in the control of development in B. subtilis. Cell 79, 1047-1055 (1994)]. In contrast to Bacilli, Paenibacillus species are lacking the sporulation repressor RapA as prominent gene orchestrating a heterochronic sporulation on single-cell level.

Furthermore, under nutrient starvation, CodY regulate the expression of many genes in Bacillus coordinating the transition from rapid exponential growth to stationary phase and sporulation. (Ratnayake-Lecamwasam M, Serror P, Wong K W, Sonenshein A L. Bacillus subtilis CodY represses early-stationary-phase genes by sensing GTP levels. Genes Dev. 2001; 15(9):1093-1103. doi:10.1101/gad.874201). This can be supported by quorum-sensing activity of ComA coordinating inter-species communication, differentiation or synchronization in cultivations (Schultz D, Wolynes P G, Ben Jacob E, Onuchic J N. Deciding fate in adverse times: sporulation and competence in Bacillus subtilis. Proc Natl Acad Sci USA. 2009 Dec. 15; 106(50):21027-34. doi: 10.1073/pnas.0912185106. Epub 2009 Dec. 7). Here again, in contrast to Bacillus, entry into sporulation of Paenibacillus is dependent on different mechanisms since CodY and ComA were not found in most Paenibacillus species.

Despite being an object of research for a long time, it has only recently been found that endospores in Bacillus subtilis come in two varieties, i.e. so called early and late spores. The authors of the publication Mutlu et al., Nature Comm. 2018, 69, have monitored sporulation and germination of Bacillus subtilis colonies on agarose plates. They recorded, for each spore formed, the time required for spore formation after nutrient downshift. After 4 days of starvation, spore formation and release of spores from sporangia was completed. A nutrient upshift was applied to the agarose plates. The authors then correlated the time each spore required for germination and outgrowth. When correlating spore formation and germination times, the authors noted that early spores germinated twice as fast as late spores and had a higher overall revival frequency. The authors also noticed that in contrast to early spores, late spores could be prevented from outgrowth by inducing germination at a nutrient concentration unsuitable for outgrowth.

The present invention relies on further observations. The inventors surprisingly noticed that different endospore community types, i.e. endospore communities differing in germination frequency and germination time, are produced according to spore formation time for all tested species of endospore forming microorganisms. This was particularly surprising, because the aforementioned publication by Mutlu et al. relied on the functional expression of the RapA gene, which is absent for example in Paenibacillus species. Thus, it was unexpected that a particular sporulation mechanism established in Bacillus subtilis would also be present in other genera. Furthermore the inventors noticed that the difference between endospore community types is not confined to spore formation on agarose plates but also occurs in stirred fermentations. This was particularly surprising because in stirred fermentation inter-cell communication gradient formation of chemical signals and nutrients is not possible. In addition, local nutrient competition, unfavorable pH changes and local waste accumulation are impossible under controlled and stirred conditions. Under optimal stirring conditions, all cells are exposed to nearly the same medium composition. It was also surprising to the inventors that endospores communities having a high germination frequency and a short germination time can also be stored extensively without significant loss of activity under normal storage conditions, like temperatures of −80° C. to 45° C. This was particularly surprising because the inventors also found that dipicolinic acid, a compound required for spore stabilization, is mainly produced late during liquid phase stirred fermentation. Thus, endospores formed early during fermentation contain a low content of dipicolinic acid. The inventors also surprisingly observed that the length of the lag phase and the time required to reach the end of log phase biomass production in liquid stirred fermentations depends on the endospore community type used as seed for inoculation of the preculture and did also positively affect growth and productivity in main culture stage. This was surprising because endospore communities harvested late during a stirred liquid phase fermentation comprise all spores formed early during fermentation. It was thus to be expected that late harvested endospore communities would at least not lag behind endospore communities harvested early during fermentation. This expectation was corroborated by the above publication by Mutlu et al., which describes differences in germination time for a completely sporulated colony of Bacillus subtilis—this would correspond, essentially, to an endospore community harvested after full sporulation of all vegetative cells in a stirred liquid phase fermentation. And the inventors found that surprisingly the content of plant beneficial biopesticides, most notably fusaricidins A, B and D, is highest in endospore communities produced early during fermentation if early spores were exclusively used as seed for inoculation of cultivations.

It was thus an object of the present invention to provide endospore containing compositions which promote early germination and rapid growth of the germinated microorganisms. Rapid outgrowth of spores is in particularly relevant for products whose performance is strongly linked to a fast and reliable outgrowth of the spores to obtain the desired properties and traits of the organism in a timely and continuous manner. Furthermore, the compositions should be stable under normal storage conditions. Preferably the compositions should maintain or improve the microorganisms' beneficial properties, e.g. health benefits for humans, animals or plants or the production of desired metabolites. The invention furthermore should provide corresponding production methods, products and uses thereof.

### BRIEF SUMMARY

The invention correspondingly provides a spore composition comprising purified spores of a prokaryotic microorganism, wherein
a) said spores form colonies when plated on a medium suitable for colony formation, and wherein of all such colonies formed within 72 h for aerobic cultures and 96 h for anaerobic cultures after plating at least 40% are formed within 48 h, more preferably 40-90%, more preferably at least 50%, more preferably 50-90%, more preferably at least 60%, more preferably 60-90%, more preferably at least 70%, more preferably 70-90%, and/or
b) at least 40% of spores are obtainable or obtained from a fermentation harvested during a first spore formation phase, more preferably at least 50%, more preferably at least 55%, more preferably at least 60%, more preferably at least 70%, more preferably at least 80% and/or
c) the mean content of dipicolinic acid per spore is at most 80% of the mean content of dipicolinic acid of spores fermented in a suitable medium until plateau phase, more preferably 20-80%, even more preferably 22-70%, even more preferably 30-65%.

The invention also provides a plant protection product, comprising a plant cultivation substrate coated or infused with a composition of the invention or obtainable or obtained by a method according to the invention.

Also provided is a plant, plant part or plant propagation material, wherein the material comprises, on its surface or infused therein, a composition according to the invention or obtainable or obtained by a method according to the invention.

Furthermore, the invention provides a plantation, preferably a field or a greenhouse bed, comprising a plant, plant part or plant propagation material of the invention or a plant cultivation substrate of the invention.

The invention also provides a food or feed or cosmetic product comprising a composition of the present invention, preferably a probiotic or prebiotic food, a probiotic or prebiotic or feed or a probiotic or prebiotic cosmetic product.

And the invention provides a building product comprising a composition according to the invention, preferably a paint, coat or impregnation composition for the treatment of mineral surfaces, a cement formulation, an additive for the preparation of a concrete or a set concrete.

Furthermore, the invention provides a method of producing a composition comprising spores of a prokaryotic microorganism, comprising the steps of
1) fermenting the microorganism in a medium conductive to sporulation,
2) purifying the spores to obtain the composition,
   wherein
a) purification is performed latest when 85% of the maximum spore concentration obtainable in the fermentation step 1) is reached, more preferably purification is performed when a concentration in the range of 1-75% relative to said maximum is reached, more preferably when a concentration in the range of 10-75% relative to said maximum is reached, more preferably when a concentration in the range of 20-70% relative to said maximum is reached, more preferably when a concentration in the range of 30-68% relative to said maximum is reached, and/or
b) purification is performed such that said purified spores form colonies when plated on a medium suitable for colony formation, and wherein of all such colonies formed within 72 h for aerobic cultures and 96 h for anaerobic cultures after plating at least 40% are formed within 48 h, more preferably 40-90%, more preferably at least 50%, more preferably 50-90%, more preferably at least 60%, more preferably 60-90%, more preferably at least 70%, more preferably 70-90%, and/or
c) purification is performed such that said purified at least 40% of spores are obtainable or obtained from a fermentation harvested during a first spore formation phase, more preferably at least 50%, more preferably at least 55%, more preferably at least 60%, more preferably at least 70%, more preferably at least 80% and/or
d) purification is performed when the mean content of dipicolinic acid per spore is at most 80% of the mean content of dipicolinic acid of spores produced when reaching maximum spore concentration in the fermentation step 1), more preferably the mean content of dipicolinic acid is in the range of 20-80%, even more preferably in the range of 22-70%, even more preferably in the range of 30-65%.

Correspondingly, the invention provides a fermentation method, comprising the step of inoculating a fermenter comprising a suitable fermentation medium with a composition of the invention or obtainable or obtained by a method according to the invention.

The invention furthermore provides a method for controlling, in a fermentation of spore-forming prokaryotic microorganisms, the duration of a lag phase and/or the time until reaching the end of log phase, comprising inoculating a suitable fermentation medium with a composition of the invention or obtainable or obtained by a method according to the invention and fermenting the inoculated medium, wherein for shorter duration of the lag phase and/or faster end of log phase a composition is used having a higher percentage of spores harvested in a first spore formation phase, and for longer duration of lag phase or later end of log phase a composition is used having a higher percentage of spores harvested in a second spore formation phase.

And the invention provides a computer-implemented method for providing an inoculant sample for fermentation, comprising the steps of
i) obtaining a target duration of the lag phase and/or end of log phase,
ii) calculating the required percentage of spores harvested during the first spore formation phase and/or the second spore formation phase, and
iii) performing a reaction based on the calculation in step 2 selected from one or more of:
(1) emission of an identifier of an inoculant sample of a working cell bank sample collection best fitting to the calculated ratio,
(2) retrieval of an inoculant sample of a working cell bank sample collection best fitting to the calculated ratio,
(3) dosing of an inoculant sample of a working cell bank sample collection best fitting to the calculated ratio to the fermenter, or
(4) mixing of a new working cell bank sample by adjusting the proportion of early and late spore communities by drawing from an early spore community enriched and from a late spore community enriched stock, respectively, and optionally dosing said mixture to the fermenter.

Furthermore provided by the invention is a method of promoting spore germination and/or vegetative growth of a spore-forming prokaryotic microorganism, comprising providing spores harvested during a first spore formation phase in a method according to the invention, wherein preferably inorganic phosphate is provided together or sequentially with the spores.

The invention also teaches a use of a composition of the invention or obtainable or obtained by a method according to the invention
a) for inoculating a fermentation, or
b) for pest control and/or for preventing, delaying, limiting or reducing the intensity of a phytopathogenic fungal or bacterial disease and/or for improving the health of a plant and/or for increasing yield of plants and/of for preventing, delaying, limiting or reducing the emission of phytopathogenic fungal or bacterial material from a plant cultivation area, or
c) for the preparation of a plant protection product, or
d) for the preparation of a probiotic food, feed or cosmetic formulation, or
e) for the preparation of a cleaning product, preferably for imparting, increasing or prolonging an antibacterial or antifungal effect of a cleaning product,
e) for the preparation of a concrete or for painting, coating or impregnating a mineral surface.

Also provided according to the invention is a method of protecting a plant or part thereof in need of protection from pest damage, comprising contacting the pest, plant, a part or propagation material thereof or to the substrate where the plants are to grow with an effective amount of a composition of the invention or obtainable or obtained by a method according to the invention, preferably before or after planting, before or after emergence, or preferably as particulates, a powder, suspension or solution.

Furthermore, the invention provides a method of delivering a protein payload to a plant, plant part, seed or growth substrate, comprising applying a composition of the invention or obtainable or obtained by a method according to the invention to the plant, plant part, seed or substrate, wherein the spores are those of a microorganism expressing a protein comprising a payload domain and a targeting domain for delivery of the payload domain to the surface of said spores.

And the invention provides a particular use or method according to the invention, wherein
i) the fungal disease is selected from white blister, downy mildews, powdery mildews, clubroot, sclerotinia rot, fusarium wilts and rots, botrytis rots, anthracnose, rhizoctonia rots, damping-off, cavity spot, tuber diseases, rusts, black root rot, target spot, aphanomyces root rot, ascochyta collar rot, gummy stem blight, alternaria leaf spot, black leg, ring spot, late blight, cercospora, leaf blight, septoria spot, leaf blight, or a combination thereof, and/or
ii) the fungal disease is caused or aggravated by a microorganism selected from the taxonomic ranks:
   class Sordariomycetes, more preferably of order Hypocreales, more preferably of family Nectriaceae, more preferably of genus Fusarium;
   class Sordariomycetes, more preferably of order Glomerellales, more preferably of family Glomerellaceae, more preferably of genus Colletotrichum:
   class Leotinomycetes, more preferably of order Helotiales, more preferably of family Sclerotiniaceae, more preferably of genus Botrytis;
   class Dothideomycetes, more preferably of order Pleosporales, more preferably of family Pleosporaceae, more preferably of genus Alternaria;
   class Dothideomycetes, more preferably of order Pleosporales, more preferably of family Phaeosphaeriaceae, more preferably of genus Phaeosphaeria;
   class Dothideomycetes, more preferably of order Botryosphaeriales, more preferably of family Botryosphaeriaceae, more preferably of genus Macrophomina;
   class Dothideomycetes, more preferably of order Capnodiales, more preferably of family Mycosphaerellaceae, more preferably of genus Zymoseptoria;
   class Agraricomycetes, more preferably of order Cantharellales, more preferably of family Ceratobasidiaceae, more preferably of genus Rhizoctonia or Thanatephorus;
   class Pucciniomycetes, more preferably of order Pucciniales, more preferably of family Pucciniaceae, more preferably of genus Uromyces or Puccinia;
   class Ustilaginomycetes, more preferably of order Ustilaginales, more preferably of family Ustilaginaceae, more preferably of genus Ustilago;
   class Oomycota, more preferably of order Pythiales, more preferably of family Pythiaceae, more preferably of genus Pythium;
   class Oomycota, more preferably of order Peronosporales, more preferably of family Peronosporaceae, more preferably of genus Phytophthora, Plasmopara or Pseudoperonospora.

### BRIEF DESCRIPTION OF THE DRAWINGS

FIG. 1 shows the concentration of spores per ml over the course of fermentation described in example 1 using Paenibacillus strain STRAIN 32 in PX-141 medium. Spore number was assessed by phase-contrast microscopy using disposable counting chambers. The spore concentration increases in a roughly sigmoidal manner from 0 to approximately 3.5×10\hat{ }9. Spore formation, as indicated by the slope of the concentration curve, is fastest in the period of 24-30 h after inoculation and 36-42 h and lower in the period of 30-36 h.

FIG. 2 shows the number of spores produced per time interval in the fermentation described in example 1. Bar height indicates the number of spores which were produced at a specific time point. The net production of spores in each sample was determined by the following equation: NPt=Nt−Nt−1. NP=Net production of spores, N=Number of spores, t=Time point. The figure corroborates the finding of FIG. 1 that spore formation is fastest in the period of 24-30 h after inoculation and 36-42 h and lower in the period of 30-36 h.

FIG. 3 shows the development of biomass formation (in arbitrary units as measured by optical density) during fermentations inoculated with 10\hat{ }6 spores harvested in a previous fermentation in identical medium at 30, 36, 48 or 72 h fermentation time, respectively. The biomass development of all fermentations is roughly parallel, the biomass development curves are offset against each other by the length of the initial lag phase. The later the harvest time of the inoculum, the longer the lag phase and the later the end of log phase growth after inoculation.

FIG. 4 shows the time required for the fermentation of FIG. 3 to reach a biomass of ≥1 A.U, using an inoculum of 10E+6 spores harvested at 30, 36, 48 or 72 h fermentation time, respectively. The times depicted in FIG. 4 indicate the length of the lag phase. The later the harvest time of the inoculum, the longer the lag phase.

FIG. 5 shows the total fusaricidin A, B and D concentration after 48 h cultivation time using 10E+6 spores/ml as initial inoculum. The spore samples used for inoculum were taken after different point in time during the 121 scale fermentation of example 1. Total fusaricidin A, B and D concentration after 48 h of fermentation was highest for a fermentation inoculated with a spore community harvested after 24 h (140%, approx. 3.5 g/l) and decreased approximately linearly with increasing inoculum harvest time, to 100% of a fermentation inoculated with a spore community harvested at 48 h. For fermentations inoculated with spore communities harvested after 48 h the decrease in total fusaricidin concentration was still measurable but not as steep as for earlier timepoints.

FIG. 6 shows spore outgrowth timing of spores harvested after 36 h and 56 h fermentation time. Colony forming units were evaluated after 48 h and 72 h cultivation time on ISP2 agar plates. Vegetative cells in fermentation broth samples were killed by heat treatment at 60° C./30 min before plating 100 μl sample on the agar plate. For spores harvested at 36 h, approximately 77% of all colonies observed within 72 h of agar plate cultivation were apparent already at 48 h of cultivation. For spores harvested at 56 h, approximately 49% of all colonies observed within 72 h of cultivation were apparent already at 48 h of cultivation.

FIG. 7 shows the viable spore titer and total dipicolinic acid level/ml fermentation broth. Samples were taken over the course of the fermentation carried out in example 4. The concentration of dipicolinic acid increases markedly faster than the speed of spore formation after approximately 40 h of fermentation.

FIG. 8 shows the development of dipicolinic acid formation normalized on spore counts. The ratio of DPA per single spore in the fermentation of example 4 was calculated as DPA [μmol/ml fermentation broth]/spore count [number/ml fermentation broth]. The concentration per spore of DPA increases fastest in the time of 40-48 h after inoculation, the highest concentration of DPA per spore was reached at 56 h of fermentation.

FIG. 9 shows outgrowth timing of spores maintained from a 7d cultivation of example 9 of C. tetanomorphum DSM528 and C. tyrobutyricum DSM1460 in TSB broth. Colony forming units were evaluated by plating 100 μl of liquid culture samples on TSB agar and visual counting after 48 h and 96 h cultivation time. Ratios of CFU found after 48 h and 96 h cultivation time relating to the total CFU counts from 96 h are shown.

### DETAILED DESCRIPTION

The technical teaching of the invention is expressed herein using the means of language, in particular by use of scientific and technical terms. However, the skilled person understands that the means of language, detailed and precise as they may be, can only approximate the full content of the technical teaching, if only because there are multiple ways of expressing a teaching, each necessarily failing to completely express all conceptual connections, as each expression necessarily must come to an end. With this in mind the skilled person understands that the subject matter of the invention is the sum of the individual technical concepts signified herein or expressed, necessarily in a pars-pro-toto way, by the innate constrains of a written description. In particular, the skilled person will understand that the signification of individual technical concepts is done herein as an abbreviation of spelling out each possible combination of concepts as far as technically sensible, such that for example the disclosure of three concepts or embodiments A, B and C are a shorthand notation of the concepts A+B, A+C, B+C, A+B+C. In particular, fallback positions for features are described herein in terms of lists of converging alternatives or instantiations. Unless stated otherwise, the invention described herein comprises any combination of such alternatives. The choice of more or less preferred elements from such lists is part of the invention and is due to the skilled person's preference for a minimum degree of realization of the advantage or advantages conveyed by the respective features. Such multiple combined instantiations represent the adequately preferred form(s) of the invention.

As used herein, terms in the singular and the singular forms like “a”, “an” and “the” include plural referents unless the content clearly dictates otherwise. Thus, for example, use of the term “a nucleic acid” optionally includes, as a practical matter, many copies of that nucleic acid molecule; similarly, the term “probe” optionally (and typically) encompasses many similar or identical probe molecules. Also as used herein, the word “comprising” or variations such as “comprises” or “comprising” will be understood to imply the inclusion of a stated element, integer or step, or group of elements, integers or steps, but not the exclusion of any other element, integer or step, or group of elements, integers or steps.

As used herein, the term “and/or” refers to and encompasses any and all possible combinations of one or more of the associated listed items, as well as the lack of combinations when interpreted in the alternative (“or”). The term “comprising” also encompasses the term “consisting of”.

The term “about”, when used in reference to a measurable value, for example an amount of mass, dose, time, temperature, sequence identity and the like, refers to a variation of 0.1%, 0.25%, 0.5%, 0.75%, 1%, 2%, 3%, 4%, 5%, 6%, 7%, 8%, 9%, 10%, 15% or even 20% of the specified value as well as the specified value. Thus, if a given composition is described as comprising “about 50% X,” it is to be understood that, in some embodiments, the composition comprises 50% X whilst in other embodiments it may comprise anywhere from 40% to 60% X (i.e., 50%±10%).

The term “plant” is used herein in its broadest sense as it pertains to organic material and is intended to encompass eukaryotic organisms that are members of the taxonomic kingdom plantae, examples of which include but are not limited to monocotyledon and dicotyledon plants, vascular plants, vegetables, grains, flowers, trees, herbs, bushes, grasses, vines, ferns, mosses, fungi and algae, etc, as well as clones, offsets, and parts of plants used for asexual propagation (e.g. cuttings, pipings, shoots, rhizomes, underground stems, clumps, crowns, bulbs, corms, tubers, rhizomes, plants/tissues produced in tissue culture, etc.). Unless stated otherwise, the term “plant” refers to a whole plant, any part thereof, or a cell or tissue culture derived from a plant, comprising any of: whole plants, plant components or organs (e.g., leaves, stems, roots, etc.), plant tissues, seeds, plant cells, and/or progeny of the same. A plant cell is a biological cell of a plant, taken from a plant or derived through culture from a cell taken from a plant.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp., Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amaranth, artichoke, asparagus, broccoli, Brussels sprouts, cabbage, canola, carrot, cauliflower, celery, collard greens, flax, kale, lentil, oilseed rape, okra, onion, potato, rice, soybean, strawberry, sugar beet, sugar cane, sunflower, tomato, squash, tea and algae, amongst others. According to a preferred embodiment of the present invention, the plant is a crop plant. Examples of crop plants include inter alia soybean, sunflower, canola, alfalfa, rapeseed, cotton, tomato, potato or tobacco.

According to the invention, a plant is cultivated to yield plant material. Cultivation conditions are chosen in view of the plant and may include, for example, any of growth in a greenhouse, growth on a field, growth in hydroculture and hydroponic growth. Plants and plant parts, for example seeds and cells, can be genetically modified. In particular, plants and parts thereof, preferably seed and cells, can be recombinant, preferably transgenic or cisgenic.

The present invention provides a spore composition. According to the invention, the terms “spore” and “endospore” are used interchangeably. The terms include both germinatable spores and non-germinatable spores, i.e. spore bodies not containing viable microorganism material or genetic modifications preventing further germination or outgrowth. Spore bodies comprise an outer layer which typically acts as a semipermeable barrier to the environment and relays chemical signals of the environment to the cell material within the spore, for example to trigger germination. The outer layer is typically further divided into an exosporium and a coat. The spore outer layer is thus an object of research in its own right and has been analysed extensively for Bacillus, Clostridium (Abhyankar et al., J Proteome Res. 2013, 4507-4521) and Paenibacillus (WO2020232316). The core of the spore comprises a complex of calcium-dipicolinic acid (DPA) that contributes up to 4-15% of the spore's dry weight (Church, B., Halvorson, H. Dependence of the Heat Resistance of Bacterial Endospores on their Dipicolinic Acid Content. Nature 183, 124-125 (1959). https://doi.org/10.1038/183124a0). Dipicolinic acid has been found out to bind free water molecules causing dehydration of the spore and thus improving the heat resistance of macromolecules within the core (I. Smith, R. Slepecky, P. Setlow, Gerhardt, P., 1989 Spore thermoresistance mechanisms. In Regulation of Procaryotic Development, edited by I. Smith, R. Slepecky, and P. Setlow, pp. 17-37, American Society for Microbiology, Washington, D.C). In addition, the calcium-dipicolinic acid complex protects DNA from heat denaturation by inserting itself between the nucleobases and thus increasing the stability of DNA (Moeller, R., M. Raguse, G. Reitz, R. Okayasu, Z. Li, et al., 2014 Resistance of Bacillus subtilis spore dna to lethal ionizing radiation damage relies primarily on spore core components and dna repair, with minor effects of oxygen radical detoxification. Applied and Environmental Microbiology 80: 104-109). Preferably the term spore indicates a viable, i.e. germinatable endospore.

The spores of the spore composition are spores of a prokaryotic microorganism. Thus, the present invention does not pertain to fungal spores. Preferred taxa of prokaryotic microorganisms are described herein below.

The composition may comprise spores of several microorganism species, wherein at least one species' spores comprise a sufficient content of an early spore community as described herein, more preferably two species' spores and most preferably all spores of prokaryotic microorganisms comprise a sufficient content of a respective early spore community as described herein. The present invention correspondingly describes features to characterize a sufficient content of early spore communities in a spore composition:

Preferably, a sufficient content of early spore communities can be detected by the observation that the spores of the composition form colonies when plated on a medium suitable under appropriate conditions for colony formation. Such growth conditions and solid media are part of the skilled person's general knowledge. For example, well known media for Bacillus cultivation are M9 minimal medium (Harwood et al., 1990, Chemically defined growth media and supplements, p. 548. In C. R. Harwood and S. M. Cutting (ed.), Molecular biological methods for Bacillus. Wiley, Chichester, United Kingdom) and peptone meat extract (Naveke et al., Einführung in die mikrobiologischen Methoden, Technische Universitst Braunschweig 1982), tryptic soy broth (TSB) and Luria-Bertani (LB) (Park, C. Effect of Tryptic Soy Broth (TSB) and Luria-Bertani (LB) Medium on Production of Subtilisin CP-1 from Bacillus sp. CP-1 and Characterization of Subtilisin CP-1. Journal of Life Science (2012), 22(6), 10.5352/JLS.2012.22.6.823). After plating, colonies form at different times. According to the invention, colony formation is monitored for 72 h for aerobic cultures (30-37° C.) and 96 h for anaerobic cultures (28-35° C.) after plating the strains. Of all colonies observed within 72 h or 96 h, respectively, at least 40% have formed within 48 h for a composition according to the invention. Preferably at most 20% of all colonies will have formed after 48 h, more preferably at most 10%. Thus, preferably 40-90% of all colonies observed by the unaided eye within 72 h or 96 h, as applicable, will have formed within 48 h after cultivation. More preferably, at least 50% of the colonies will have formed within 48 h, more preferably 50-90%. Even more preferably, at least 60% of the colonies will have formed within 48 h, more preferably 60-90%. Even more preferably, at least 70% of the colonies will have formed within 48 h, more preferably 70-90%. The skilled person is aware of the fact that germination speed is to a large extent species specific. Thus, colonies may form even after 72 h/96 h of incubation. However, for the purposes of detection it is sufficient to show that the ratio of early germinating spores to later germinating spores is indeed shifted in favour of the former spore community. For example, as shown in example 10 and FIG. 10 different strains in genus Clostridium have an innate lower growth speed than, for example, Paenibacillus strains.

The composition according to the invention is preferably obtainable or obtained by purification from a fermentation, preferably a stirred liquid phase fermentation. Preferably, at least 40% of spores in a spore composition according to the invention are obtainable or obtained by purification during a first spore formation phase, more preferably at least 50%, more preferably at least 55%, more preferably at least 60%, more preferably at least 70%, more preferably at least 80%. Preferred methods of purification are described hereinafter. The end of the first spore formation phase is typically detectable by a decrease in spore formation speed. The first spore formation phase is then defined to end at the midpoint of the period of slower spore formation. However, for some fermentation media the end of the first spore formation phase may not be discernible based on the spore formation rate alone. In such cases the skilled person will perform a calibration fermentation, take samples at several points in time and determine the ratio of colony formation speeds and/or the content of dipicolinic acid per spore as described above.

The composition according to the invention preferably comprises dipicolinic acid such that the mean content of dipicolinic acid per spore is at most 80% of the mean content of dipicolinic acid of spores fermented in appropriate medium until plateau phase, more preferably 20-80%, even more preferably 22-70%, even more preferably 30-65%. As described below in more detail, the content of dipicolinic acid content per spore is advisably determined by a calibration fermentation and measurement of dipicolinic acid content in the spores and viable spore count at various times during the fermentation. When the maximum ratio of dipicolinic acid per spore is reached, it is straightforward to calculate the fermentation time when the desired dipicolinic acid content per spore is achieved.

As described herein, the composition of the present invention provides several advantages. In particular, the compositions allow for a consistent and rapid germination and outgrowth of viable spores. Furthermore the invention allows to shorten the fermentation times for preparing more active spore compositions. This is of particular interest in the industrial production of spore compositions for agriculture, probiotics and cleaning products, because a shorter production time increases the production capacity per time. It is a further advantage of the composition of the present invention that the spores in said composition, even though they belong largely to an early spore community, are nevertheless stable during extensive storage without significant loss of activity under normal storage conditions, like temperatures of −80° C. to 37° C. It was also unexpected that spore compositions of the present invention comprising Paenibacillus spores will lead to a very high productivity of fusaricidins when used to inoculate a liquid phase fermentation. Further benefits and advantages of the present invention are also described in the examples below.

The spores of the spore composition according to the present invention are purified. As described below in further detail, purification results in a suppression or reduction of spore germination in the composition as such. Generally, purification of spores entails separating spores from the fermentation medium used to cultivate the respective microorganism.

Preferably the composition comprises at most a low content of easily fermentable carbon sources. In particular it is preferred that the soluble carbon source content of the composition is at most 7% by weight of the composition, more preferably 0.1-4% by weight of the composition.

Furthermore, the water content of the composition is preferably adjusted to at most 98% by weight of the composition in liquid formulations, more preferably 80-95% by weight of the composition. In dry formulations, the water content of the composition is preferably adjusted to at most 10% by weight of the composition in liquid formulations, more preferably 2-8% by weight of the composition Preferably purification comprises concentrating of spores and preferably comprises a step of desiccation, lyophilization, homogenization, extraction, tangential flow filtration, depth filtration, centrifugation or sedimentation. Such methods of downstream processing are generally known to the skilled person, they can be performed using standard industry equipment and using minimal adaptation of methods known in the art. It is thus a particular advantage of the present invention that the compositions of the present invention can easily be produced at low costs.

Correspondingly the spore composition of the present invention preferably comprises viable cells and spores in a ratio of at most 4:1, more preferably 3:1 to 0.2:1. In certain applications, a combination of viable cells enabling rapid proliferation without external triggers required for germination as well as spores allowing long-term efficacy and product stability can be beneficial. However, as described herein, the invention is mainly concerned with providing spores in compositions, the presence of viable cells is thus according to the invention tolerated but is not mandatory. Furthermore, it has been frequently observed that so-called cell-free preparations may not be devoid of cells but rather are largely cell-free or essentially cell-free, depending on the technique used (e.g., speed of centrifugation) to remove the cells. The resulting cell-free preparation may be dried and/or formulated with components that aid in its application to plants or to plant growth media. It is an advantage of the present invention that the compositions can tolerate the presence of cells, including cells of the prokaryotic microorganism(s) which produced the spores of the composition. On the other hand, the spore composition of the present invention can also be a composition free of viable cells.

The spore composition of the present invention preferably comprises, in addition to said spores, at least one pest control agent preferably selected from the group consisting of
i) one or more microbial pesticides with fungicidal, bactericidal, viricidal and/or plant defense activator activity,
ii) one or more biochemical pesticides with fungicidal, bactericidal, viricidal and/or plant defense activator activity,
iii) one or more microbial pesticides with insecticidal, acaricidal, molluscidal and/or nematicidal activity,
iv) one or more biochemical pesticides with insecticidal, acaricidal, molluscidal, pheromone and/or nematicidal activity,
v) one or more fungicide selected from respiration inhibitors, sterol biosynthesis inhibitors, nucleic acid synthesis inhibitors, inhibitors of cell division and cytoskeleton formation or function, inhibitors of amino acid and protein synthesis, signal transduction inhibitors, lipid and membrane synthesis inhibitors, inhibitors with multi-site action, cell wall synthesis inhibitors, plant defence inducers and fungicides with unknown mode of action.

Biopesticides fall into two major classes, microbial and biochemical pesticides. Microbial pesticides consist of bacteria, fungi or viruses and often include the metabolites that bacteria and fungi produce. Entomopathogenic nematodes are also classified as microbial pesticides, even though they are multi-cellular. Biochemical pesticides are naturally occurring substances or structurally-similar and functionally identical to a naturally-occurring substance and extracts from biological sources that control pests or provide other crop protection uses as defined below, but have non-toxic mode of actions (such as growth or developmental regulation, attractants, repellents or defense activators (e.g. induced resistance) and are relatively non-toxic to mammals. Biopesticides for use against crop diseases have already established themselves on a variety of crops. For example, biopesticides already play an important role in controlling downy mildew diseases. Their benefits include: a 0-Day Pre-Harvest Interval, the ability to use under moderate to severe disease pressure, and the ability to use in mixture or in a rotational program with other registered pesticides. It is a particular advantage of the present invention that several biopesticides are produced by spore forming prokaryotic microorganisms. Thus, the compositions and corresponding methods of the present invention not only allow for a rapid production of such biopesticides, the compositions advantageously also support a fast and successful germination of spores which either are biopesticidal on their own, e.g. by having pesticides with fungicidal, bactericidal, viricidal and/or plant defense activator activity attached to the spore's outer layer (fusaricidins being one example thereof), or which produce such biopesticides after germination. Thus, the compositions of the present invention particularly support the preparation of agricultural products comprising biopesticidal spores of prokaryotic microorganisms.

Thus, the spore composition of the present invention preferably comprises biopesticidal spores and optionally further biopesticides. Many biopesticides have been deposited under deposition numbers mentioned herein (the prefices such as ATCC or DSM refer to the acronym of the respective culture collection, for details see e. g. here: http://www. wfcc.info/ccinfo/collection/by_acronym/), are referred to in literature, registered and/or are commercially available: mixtures of Aureobasidium pullulans DSM 14940 and DSM 14941 isolated in 1989 in Konstanz, Germany (e. g. blastospores in BlossomProtect® from bio-ferm GmbH, Austria), Azospirillum brasilense Sp245 originally isolated in wheat region of South Brazil (Passo Fundo) at least prior to 1980 (BR 11005; e. g. GELFIX® Gramineas from BASF Agricultural Specialties Ltd., Brazil), A. brasilense strains Ab-V5 and Ab-V6 (e. g. in AzoMax from Novozymes BioAg Produtos papra Agricultura Ltda., Quattro Barras, Brazil or Simbiose-Maiz® from Simbiose-Agro, Brazil; Plant Soil 331, 413-425, 2010), Bacillus amyloliquefaciens strain AP-188 (NRRL B-50615 and B-50331; U.S. Pat. No. 8,445,255); B. amyloliquefaciens spp. plantarum D747 isolated from air in Kikugawashi, Japan (US 20130236522 A1; FERM BP 8234; e. g. Double Nickel™ 55 WDG from Certis LLC, USA), B. amyloliquefaciens spp. plantarum FZB24 isolated from soil in Brandenburg, Germany (also called SB3615; DSM 96-2; J. Plant Dis. Prot. 105, 181-197, 1998; e. g. Taegro® from Novozyme Biologicals, Inc., USA), B. amyloliquefaciens ssp. plantarum FZB42 isolated from soil in Brandenburg, Germany (DSM 23117; J. Plant Dis. Prot. 105, 181-197, 1998; e. g. RhizoVital® 42 from AbiTEP GmbH, Germany), B. amyloliquefaciens ssp. plantarum MB1600 isolated from faba bean in Sutton Bonington, Nottinghamshire, U.K. at least before 1988 (also called 1430; NRRL B 50595; US 2012/0149571 A1; e. g. Integral® from BASF Corp., USA), B. amyloliquefaciens spp. plantarum QST-713 isolated from peach orchard in 1995 in California, U.S.A. (NRRL B 21661; e. g. Serenade® MAX from Bayer Crop Science LP, USA), B. amyloliquefaciens spp. plantarum TJ1000 isolated in 1992 in South Dakoda, U.S.A. (also called 1 BE; ATCC BAA-390; CA 2471555 A1; e. g. QuickRoots™ from TJ Technologies, Watertown, SD, USA), B. firmus CNCM I-1582, a variant of parental strain EIP-N1 (CNCM I-1556) isolated from soil of central plain area of Israel (WO 2009/126473, U.S. Pat. No. 6,406,690; e. g. Votivo® from Bayer CropScience LP, USA), B. pumilus GHA 180 isolated from apple tree rhizosphere in Mexico (IDAC 260707-01; e. g. PRO-MIX® BX from Premier Horticulture, Quebec, Canada), B. pumilus INR-7 otherwise referred to as BU F22 and BU-F33 isolated at least before 1993 from cucumber infested by Erwinia tracheiphila (NRRL B-50185, NRRL B-50153; U.S. Pat. No. 8,445,255), (NRRL B-50754; WO 2014/029697; B. pumilus QST 2808 was isolated from soil collected in Pohnpei, Federated States of Micronesia, in 1998 (NRRL B 30087; e. g. Sonata® or Ballad® Plus from Bayer Crop Science LP, USA), B. simplex ABU 288 (NRRL B-50304; U.S. Pat. No. 8,445,255), B. subtilis FB17 also called UD 1022 or UD10-22 isolated from red beet roots in North America (ATCC PTA-11857; System. Appl. Microbiol. 27, 372-379, 2004; US 2010/0260735; WO 2011/109395); B. thuringiensis ssp. aizawai ABTS-1857 isolated from soil taken from a lawn in Ephraim, Wisconsin, U.S.A., in 1987 (also called ABG 6346; ATCC SD-1372; e. g. XenTari® from BioFa AG, Münsingen, Germany), B. t. ssp. kurstaki ABTS-351 identical to HD-1 isolated in 1967 from diseased Pink Bollworm black larvae in Brownsville, Texas, U.S.A. (ATCC SD-1275; e. g. Dipel® DF from Valent BioSciences, IL, USA), B. t. ssp. kurstaki SB4 isolated from E. saccharina larval cadavers (NRRL B-50753; B. t. ssp. tenebrionis NB-176-1, a mutant of strain NB-125, a wild type strain isolated in 1982 from a dead pupa of the beetle Tenebrio molitor (DSM 5480; EP 585 215 B1; e. g. Novodor® from Valent BioSciences, Switzerland), Beauveria bassiana GHA (ATCC 74250; e. g. BotaniGard® 22WGP from Laverlam Int. Corp., USA), B. bassiana JW-1 (ATCC 74040; e. g. Naturalis® from CBC (Europe) S.r.l., Italy), B. bassiana PPRI 5339 isolated from the larva of the tortoise beetle Conchyloctenia punctata (NRRL 50757), Bradyrhizobium elkanii strains SEMIA 5019 (also called 29W) isolated in Rio de Janeiro, Brazil and SEMIA 587 isolated in 1967 in the State of Rio Grande do Sul, from an area previously inoculated with a North American isolate, and used in commercial inoculants since 1968 (Appl. Environ. Microbiol. 73(8), 2635, 2007; e. g. GELFIX 5 from BASF Agricultural Specialties Ltd., Brazil), B. japonicum 532c isolated from Wisconsin field in U.S.A. (Nitragin 61A152; Can. J. Plant. Sci. 70, 661-666, 1990; e. g. in Rhizoflo®, Histick®, Hicoat® Super from BASF Agricultural Specialties Ltd., Canada), B. japonicum E-109 variant of strain USDA 138 (INTA E109, SEMIA 5085; Eur. J. Soil Biol. 45, 28-35, 2009; Biol. Fertil. Soils 47, 81-89, 2011); B. japonicum strains deposited at SEMIA known from Appl. Environ. Microbiol. 73(8), 2635, 2007: SEMIA 5079 isolated from soil in Cerrados region, Brazil by Embrapa-Cerrados used in commercial inoculants since 1992 (CPAC 15; e. g. GELFIX 5 or ADHERE 60 from BASF Agricultural Specialties Ltd., Brazil), B. japonicum SEMIA 5080 obtained under lab conditions by Embrapa-Cerrados in Brazil and used in commercial inoculants since 1992, being a natural variant of SEMIA 586 (CB1809) originally isolated in U.S.A. (CPAC 7; e. g. GELFIX 5 or AD-HERE 60 from BASF Agricultural Specialties Ltd., Brazil); Burkholderia sp. A396 isolated from soil in Nikko, Japan, in 2008 (NRRL B-50319; WO 2013/032693; Marrone Bio Innovations, Inc., USA), Coniothyrium minitans CON/M/91-08 isolated from oilseed rape (WO 1996/021358; DSM 9660; e. g. Contans® WG, Intercept® WG from Bayer CropScience AG, Germany), harpin (alpha-beta) protein (Science 257, 85-88, 1992; e. g. Messenger™ or HARP-N Tek from Plant Health Care plc, U.K.), Helicoverpa armigera nucleopolyhedrovirus (HearNPV) (J. Invertebrate Pathol. 107, 112-126, 2011; e. g. Helicovex® from Adermatt Biocontrol, Switzerland; Diplomata® from Koppert, Brazil; Vivus® Max from AgBiTech Pty Ltd., Queensland, Australia), Helicoverpa zea single capsid nucleopolyhedrovirus (HzSNPV) (e. g. Gemstar® from Certis LLC, USA), Helicoverpa zea nucleopolyhedrovirus ABA-NPV-U (e. g. Heligen® from AgBiTech Pty Ltd., Queensland, Australia), Heterorhabditis bacteriophora (e. g. Nemasys® G from BASF Agricultural Specialties Limited, UK), Isaria fumosorosea Apopka-97 isolated from mealy bug on gynura in Apopka, Florida, U.S.A. (ATCC 20874; Biocontrol Science Technol. 22(7), 747-761, 2012; e. g. PFR-97™ or PreFeRal® from Certis LLC, USA), Metarhizium anisopliae var. anisopliae F52 also called 275 or V275 isolated from codling moth in Austria (DSM 3884, ATCC 90448; e. g. Met52® Novozymes Biologicals Bio-Ag Group, Canada), Metschnikowia fructicola 277 isolated from grapes in the central part of Israel (U.S. Pat. No. 6,994,849; NRRL Y-30752; e. g. formerly Shemer® from Agrogreen, Israel), Paecilomyces ilacinus 251 isolated from infected nematode eggs in the Philippines (AGAL 89/030550; WO1991/02051; Crop Protection 27, 352-361, 2008; e. g. BioAct® from Bayer CropScience AG, Germany and MeloCon® from Certis, USA), Pasteuria nishizawae Pn1 isolated from a soybean field in the mid-2000s in Illinois, U.S.A. (ATCC SD 5833; Federal Register 76(22), 5808, Feb. 2, 2011; e.g. Clariva™ PN from Syngenta Crop Protection, LLC, USA), Penicillium bilaiae (also called P. bilaii) strains ATCC 18309 (=ATCC 74319), ATCC 20851 and/or ATCC 22348 (=ATCC 74318) originally isolated from soil in Alberta, Canada (Fertilizer Res. 39, 97-103, 1994; Can. J. Plant Sci. 78(1), 91-102, 1998; U.S. Pat. No. 5,026,417, WO 1995/017806; e. g. Jump Start®, Provide® from Novozymes Biologicals BioAg Group, Canada), Reynoutria sachalinensis extract (EP 0307510 B1; e. g. Regalia® SC from Marrone BioInnovations, Davis, CA, USA or Milsana® from BioFa AG, Germany), Steinernema carpocapsae (e. g. Millenium® from BASF Agricultural Specialties Limited, UK), S. feltiae (e. g. Nemashield® from BioWorks, Inc., USA; Nemasys® from BASF Agricultural Specialties Limited, UK), Streptomyces microflavus NRRL B-50550 (WO 2014/124369; Bayer CropScience, Germany), T. harzianum T-22 also called KRL-AG2 (ATCC 20847; Bio-Control 57, 687-696, 2012; e. g. Plantshield® from BioWorks Inc., USA or SabrEx™ from Advanced Biological Marketing Inc., Van Wert, OH, USA).

The spore forming microorganism according to the present invention is preferably selected from the taxonomic rank of phylum Firmicutes, class Bacilli, Clostridia or Negativicutes, more preferably of order Bacillales, Clostridiales, Thermoanaerobacterales, Thermosediminibacterales or Selenomonadales, more preferably of family Bacillaceae, Paenibacillaceae, Pasteuriaceae, Clostridiaceae, Peptococcaceae, Heliobacteriaceae, Syntrophomonadaceae, Thermoanaerobacteraceae, Tepidanaerobacteraceae or Sporomusaceae, more preferably of genus Alkalibacillus, Bacillus, Geobacillus, Halobacillus, Lysinibacillus, Piscibacillus, Terribacillus, Brevibacillus, Paenibacillus, Thermobacillus, Pasteuria, Clostridium, Desulfotomaculum, Heliobacterium, Pelospora, Pelotomaculum, Caldanaerobacter, Moorella, Thermoanaerobacter, Tepidanaerobacter, Propionispora or Sporomusa, more preferably of genus Bacillus, Paenibacillus or Clostridium. Microorganisms of these taxa are known to the skilled person; methods for their cultivation are available and form part of the routine work of the person skilled in the art. It is advantageous that many of the aforementioned microorganisms are of industrial relevance, for example for producing relevant agricultural compositions or probiotics. In particular, microorganisms of family Bacillaceae, Paenibacillaceae and Clostridiaceae are relevant and are known to exert fungicidal and/or bactericidal effects.

Within the composition of the present invention, spores in particular of the following species are preferred:

Paenibacillus species: P. abekawaensis, P. abyssi, P. aceris, P. aceti, P. aestuarii, P. agarexedens, P. agaridevorans, P. alba, P. albidus, P. albus, P. alginolyticus, P. algorifonticola, P. alkaliterrae, P. alvei, P. amylolyticus, P. anaericanus, P. antarcticus, P. antibioticophila, P. antri, P. apiaries, P. apiarius, P. apis, P. aquistagni, P. arachidis, P. arcticus, P. assamensis, P. aurantiacus, P. azoreducens, P. azotifigens, P. baekrokdamisoli, P. barcinonensis, P. barengoltzii, P. beijingensis, P. borealis, P. bouchesdurhonensis, P. bovis, P. brasilensis, P. brassicae, P. bryophyllum, P. caespitis, P. camelliae, P. camerounensis, P. campinasensis, P. castaneae, P. catalpae, P. cathormii, P. cavernae, P. cellulosilyticus, P. cellulositrophicus, P. chartarius, P. chibensis, P. chinensis, P. chinjuensis, P. chitinolyticus, P. chondroitinus, P. chungangensis, P. cineris, P. cisolokensis, P. contaminans, P. cookii, P. crassostreae, P. cucumis, P. curdlanolyticus, P. daejeonensis, P. dakarensis, P. darangshiensis, P. darwinianus, P. dauci, P. dendritiformis, P. dongdonensis, P. donghaensis, P. doosanensis, P. durus, P. edaphicus, P. ehimensis, P. elgii, P. elymi, P. endophyticus, P. enshidis, P. esterisolvens, P. etheri, P. eucommiae, P. faecis, P. favisporus, P. ferrarius, P. filicis, P. flagellatus, P. fonticola, P. forsythiae, P. frigoriresistens, P. fujiensis, P. fukuinensis, P. gansuensis, P. gelatinilyticus, P. ginsengagri, P. ginsengarvi, P. ginsengihumi, P. ginsengiterrae, P. glacialis, P. glebae, P. glucanolyticus, P. glycanilyticus, P. gorillae, P. graminis, P. granivorans, P. guangzhouensis, P. harenae, P. helianthi, P. hemerocallicola, P. herberti, P. hispanicus, P. hodogayensis, P. hordei, P. horti, P. humicus, P. hunanensis, P. ihbetae, P. ihuae, P. ihumii, P. illinoisensis, P. insulae, P. intestini, P. jamilae, P. jilunlii, P. kobensis, P. koleovorans, P. konkukensis, P. konsidensis, P. koreensis, P. kribbensis, P. kyungheensis, P. lactis, P. lacus, P. larvae, P. lautus, P. lemnae, P. lentimorbus, P. lentus, P. liaoningensis, P. limicola, P. lupini, P. luteus, P. lutimineralis, P. macerans, P. macquariensis, P. marchantiophytorum, P. marinisediminis, P. marinum, P. massiliensis, P. maysiensis, P. medicaginis, P. mendelii, P. mesophilus, P. methanolicus, P. mobilis, P. montanisoli, P. montaniterrae, P. motobuensis, P. mucilaginosus, P. nanensis, P. naphthalenovorans, P. nasutitermitis, P. nebraskensis, P. nematophilus, P. nicotianae, P. nuruki, P. oceanisediminis, P. odorifer, P. oenotherae, P. oralis, P. oryzae, P. oryzisoli, P. ottowii, P. ourofinensis, P. pabuli, P. paeoniae, P. panacihumi, P. panacisoli, P. panaciterrae, P. paridis, P. pasadenensis, P. pectinilyticus, P. peoriae, P. periandrae, P. phocaensis, P. phoenicis, P. phyllosphaerae, P. physcomitrellae, P. pini, P. pinihumi, P. pinisoli, P. pinistramenti, P. pocheonensis, P. polymyxa, P. polysaccharolyticus, P. popilliae, P. populi, P. profundus, P. prosopidis, P. protaetiae, P. provencensis, P. psychroresistens, P. pueri, P. puernese, P. puldeungensis, P. purispatii, P. qingshengii, P. qinlingensis, P. quercus, P. radicis, P. relictisesami, P. residui, P. rhizoplanae, P. rhizoryzae, P. rhizosphaerae, P. rigui, P. ripae, P. rubinfantis, P. ruminocola, P. sabinae, P. sacheonensis, P. salinicaeni, P. sanguinis, P. sediminis, P. segetis, P. selenii, P. selenitireducens, P. senegalensis, P. senegalimassiliensis, P. seodonensis, P. septentrionalis, P. sepulcri, P. shenyangensis, P. shirakamiensis, P. shunpengii, P. siamensis, P. silagei, P. silvae, P. sinopodophylli, P. solanacearum, P. solani, P. soli, P. sonchi group, P. sophorae, P. spiritus, P. sputi, P. stellifer, P. susongensis, P. swuensis, P. taichungensis, P. taihuensis, P. taiwanensis, P. taohuashanense, P. tarimensis, P. telluris, P. tepidiphilus, P. terrae, P. terreus, P. terrigena, P. tezpurensis, P. thailandensis, P. thermoaerophilus, P. thermophilus, P. thiaminolyticus, P. tianmuensis, P. tibetensis, P. timonensis, P. translucens, P. tritici, P. triticisoli, P. tuaregi, P. tumbae, P. tundrae, P. turicensis, P. tylopili, P. typhae, P. tyrfis, P. uliginis, P. urinalis, P. validus, P. velaei, P. vini, P. vortex, P. vorticalis, P. vulneris, P. wenxiniae, P. whitsoniae, P. wooponensis, P. woosongensis, P. wulumuqiensis, P. wynnii, P. xanthanilyticus, P. xanthinilyticus, P. xerothermodurans, P. xinjiangensis, P. xylanexedens, P. xylaniclasticus, P. xylanilyticus, P. xylanisolvens, P. yanchengensis, P. yonginensis, P. yunnanensis, P. zanthoxyli, P. zeae, preferably P. agarexedens, P. agaridevorans, P. alginolyticus, P. alkaliterrae, P. alvei, P. amylolyticus, P. anaericanus, P. antarcticus, P. assamensis, P. azoreducens, P. barcinonensis, P. borealis, P. brassicae, P. campinasensis, P. chinjuensis, P. chitinolyticus, P. chondroitinus, P. cineris, P. curdlanolyticus, P. daejeonensis, P. dendritiformis, P. ehimensis, P. elgii, P. favisporus, P. glucanolyticus, P. glycanilyticus, P. graminis, P. granivorans, P. hodogayensis, P. illinoisensis, P. jamilae, P. kobensis, P. koleovorans, P. koreensis, P. kribbensis, P. lactis, P. larvae, P. lautus, P. lentimorbus, P. macerans, P. macquariensis, P. massiliensis, P. mendelii, P. motobuensis, P. naphthalenovorans, P. nematophilus, P. odorifer, P. pabuli, P. peoriae, P. phoenicis, P. phyllosphaerae, P. polymyxa, P. popilliae, P. rhizosphaerae, P. sanguinis, P. stellifer, P. taichungensis, P. terrae, P. thiaminolyticus, P. timonensis, P. tylopili, P. turicensis, P. validus, P. vortex, P. vulneris, P. wynnii, P. xylanilyticus, particularly preferred Paenibacillus koreensis, Paenibacillus rhizosphaerae, Paenibacillus polymyxa, Paenibacillus amylolyticus, Paenibacillus terrae, Paenibacillus polymyxa polymyxa, Paenibacillus polymyxa plantarum, Paenibacillus nov. spec epiphyticus, Paenibacillus terrae, Paenibacillus macerans, Paenibacillus alvei, more preferred Paenibacillus polymyxa, Paenibacillus polymyxa polymyxa, Paenibacillus polymyxa plantarum, Paenibacillus nov. spec epiphyticus, Paenibacillus terrae, Paenibacillus macerans, Paenibacillus alvei, even more preferred Paenibacillus polymyxa, Paenibacillus polymyxa polymyxa, Paenibacillus polymyxa plantarum and Paenibacillus terrae.

Bacillus species: B. abyssalis, B. acanthi, B. acidiceler, B. acidicola, B. acidiproducens, B. aciditolerans, B. acidopullulyticus, B. acidovorans, B. aeolius, B. aequororis, B. aeris, B. aerius, B. aerolacticus, B. aestuarii, B. aidingensis, B. akibai, B. alcaliinulinus, B. alcalophilus, B. algicola, B. alkalicola, B. alkalilacus, B. alkalinitrilicus, B. alkalisediminis, B. alkalitelluris, B. alkalitolerans, B. alkalogaya, B. altitudinis, B. alveayuensis, B. amiliensis, B. andreesenii, B. andreraoultii, B. aporrhoeus, B. aquimaris, B. arbutinivorans, B. aryabhattai, B. asahii, B. aurantiacus, B. australimaris, B. azotoformans, B. bacterium, B. badius, B. baekryungensis, B. bataviensis, B. benzoevorans, B. beringensis, B. berkeleyi, B. beveridgei, B. bingmayongensis, B. bogoriensis, B. borbori, B. boroniphilus, B. butanolivorans, B. cabrialesii, B. caccae, B. camelliae, B. campisalis, B. canaveralius, B. capparidis, B. carboniphilus, B. casamancensis, B. caseinilyticus, B. catenulatus, B. cavernae, B. cecembensis, B. cellulosilyticus, B. chagannorensis, B. chandigarhensis, B. cheonanensis, B. chungangensis, B. ciccensis, B. cihuensis, B. circulans, B. clausii, B. coagulans, B. coahuilensis, B. cohnii, B. composti, B. coniferum, B. coreaensis, B. crassostreae, B. crescens, B. cucumis, B. dakarensis, B. daliensis, B. danangensis, B. daqingensis, B. decisifrondis, B. decolorationis, B. depressus, B. deramificans, B. deserti, B. dielmoensis, B. djibelorensis, B. drentensis, B. ectoiniformans, B. eiseniae, B. enclensis, B. endolithicus, B. endophyticus, B. endoradicis, B. endozanthoxylicus, B. farraginis, B. fastidiosus, B. fengqiuensis, B. fermenti, B. ferrariarum, B. filamentosus, B. firmis, B. firmus, B. flavocaldarius, B. flexus, B. foraminis, B. fordii, B. formosensis, B. fortis, B. freudenreichii, B. fucosivorans, B. fumarioli, B. funiculus, B. galactosidilyticus, B. galliciensis, B. gibsonii, B. ginsenggisoli, B. ginsengihumi, B. ginsengisoli, B. glennii, B. glycinifermentans, B. gobiensis, B. gossypii, B. gottheilii, B. graminis, B. granadensis, B. hackensackii, B. haikouensis, B. halmapalus, B. halodurans, B. halosaccharovorans, B. haynesii, B. hemicellulosilyticus, B. hemicentroti, B. herbersteinensis, B. hisashii, B. horikoshii, B. horneckiae, B. horti, B. huizhouensis, B. humi, B. hunanensis, B. hwajinpoensis, B. idriensis, B. indicus, B. infantis, B. infernus, B. intermedius, B. intestinalis, B. iocasae, B. isabeliae, B. israeli, B. jeddahensis, B. jeotgali, B. kexueae, B. kiskunsagensis, B. kochii, B. kokeshiiformis, B. koreensis, B. korlensis, B. kribbensis, B. krulwichiae, B. kwashiorkori, B. kyonggiensis, B. lacisalsi, B. lacus, B. lehensis, B. lentus, B. ligniniphilus, B. lindianensis, B. litoralis, B. loiseleuriae, B. lonarensis, B. longiquaesitum, B. longisporus, B. luciferensis, B. luteolus, B. luteus, B. lycopersici, B. magaterium, B. malikii, B. mangrovensis, B. mangrovi, B. mannanilyticus, B. manusensis, B. marasmi, B. marcorestinctum, B. marinisedimentorum, B. marisflavi, B. maritimus, B. marmarensis, B. massiliglaciei, B. massilioanorexius, B. massiliogabonensis, B. massiliogorillae, B. massilionigeriensis, B. massiliosenegalensis, B. mediterraneensis, B. megaterium, B. mesonae, B. mesophilum, B. mesophilus, B. methanolicus, B. miscanthi, B. muralis, B. murimartini, B. nakamurai, B. nanhaiisediminis, B. natronophilus, B. ndiopicus, B. nealsonii, B. nematocida, B. niabensis, B. niacini, B. niameyensis, B. nitritophilus, B. notoginsengisoli, B. novalis, B. obstructivus, B. oceani, B. oceanisediminis, B. ohbensis, B. okhensis, B. okuhidensis, B. oleivorans, B. oleronius, B. olivae, B. onubensis, B. oryzae, B. oryzaecorticis, B. oryzisoli, B. oryziterrae, B. oshimensis, B. pakistanensis, B. panacisoli, B. panaciterrae, B. paraflexus, B. patagoniensis, B. persicus, B. pervagus, B. phocaeensis, B. pichinotyi, B. piscicola, B. piscis, B. plakortidis, B. pocheonensis, B. polygoni, B. polymachus, B. populi, B. praedii, B. pseudalcaliphilus, B. pseudofirmus, B. pseudoflexus, B. pseudomegaterium, B. psychrosaccharolyticus, B. pumilus, B. purgationiresistens, B. qingshengii, B. racemilacticus, B. rhizosphaerae, B. rigiliprofundi, B. rubiinfantis, B. ruris, B. safensis, B. saganii, B. salacetis, B. salarius, B. salidurans, B. salis, B. salitolerans, B. salmalaya, B. salsus, B. sediminis, B. selenatarsenatis, B. senegalensis, B. seohaeanensis, B. shacheensis, B. shackletonii, B. shandongensis, B. shivajii, B. similis, B. simplex, B. sinesaloumensis, B. siralis, B. smithii, B. solani, B. soli, B. solimangrovi, B. solisilvae, B. songklensis, B. spongiae, B. sporothermodurans, B. stamsii, B. subterraneus, B. swezeyi, B. taeanensis, B. taiwanensis, B. tamaricis, B. taxi, B. terrae, B. testis, B. thaonhiensis, B. thermoalkalophilus, B. thermoamyloliquefaciens, B. thermoamylovorans, B. thermocopriae, B. thermolactis, B. thermophilus, B. thermoproteolyticus, B. thermoterrestris, B. thermozeamaize, B. thioparans, B. tianmuensis, B. tianshenii, B. timonensis, B. tipchiralis, B. trypoxylicola, B. tuaregi, B. urumqiensis, B. vietnamensis, B. vini, B. vireti, B. viscosus, B. vitellinus, B. wakoensis, B. weihaiensis, B. wudalianchiensis, B. wuyishanensis, B. xiamenensis, B. xiaoxiensis, B. zanthoxyli, B. zeae, B. zhangzhouensis, B. zhanjiangensis,

preferably Bacillus licheniformis, B. megaterium, B. subtilis, B. pumilus, B. firmus, B. thuringiensis, B. velezensis, B. linens, B. atrophaeus, B. amyloliquefaciens, B. aryabhattai, B. cereus, B. aquatilis, B. circulans, B. clausii, B. sphaericus, B. thiaminolyticus, B. mojavensis, B. vallismortis, B. coagulans, B. sonorensis, B. halodurans, B. pocheonensis, B. gibsonii, B. acidiceler, B. flexus, B. hunanensis, B. pseudomycoides, B. simplex, B. safensis, B. mycoides, particularly preferred B. amyloliquefaciens, B. licheniformis, B. thuringiensis, B. velezensis, B. subtilis and B. megatherium,

even more preferably B. amyloliquefaciens, B. thuringiensis, B. velezensis and B. megatherium. It is a particular advantage of the present invention that the invention teaches compositions and methods of their production not only in view of Bacillus subtilis spores. In particular, the invention also provides compositions, products, methods and uses as described herein, wherein the spores do not comprise Bacillus subtilis spores but other Bacillus, Paenibacillus and/or Clostridium spores.

Clostridium species: C. autoethanogenum, C. beijerinckii, C. butyricum, C. carboxidivorans, C. disporicum, C. drakei, C. ljungdahlii, C. kluyveri, C. pasteurianum, C. propionicum, C. saccharobutylicum, C. saccharoperbutylacetonicum, C. scatologenes, C. tyrobutyricum, preferably C. butyricum, C. pasteurianum and/or C. tyrobutyricum, C. aerotolerans, C. aminophilum, C. aminvalericum, C. celerecrescens, C. asparagforme, C. bolteae, C. clostridioforme, C. glycyrrhizinilyticum, C. (Hungatela) hathewayi, C. histolyticum, C. indolis, C. leptum, C. (Tyzzerella) nexile, C. perfringens, C. (Erysipelatoclostridium) ramosum, C. scindens, C. symbiosum, Clostridium saccharogumia, Clostridium sordelli, Clostridium clostridioforme, C. methylpentosum, C. islandicum and all members of the Clostridia clusters IV, XIVa, and XVIII, particularly preferred C. butyricum.

Some suitable Bacillus and Paenibacillus strains are described and deposited in the following international patent applications; spores of such microorganisms or pesticidally active variants of any thereof can be incorporated as spores of the composition according to the invention: WO2020200959: Bacillus subtilis or Bacillus amyloliquefaciens QST713 deposited under NRRL Accession No. B-21661 or a fungicidal mutant thereof. Bacillus subtilis QST713, its mutants, its supernatants, and its lipopeptide metabolites, and methods for their use to control plant pathogens and insects are fully described in U.S. Pat. Nos. 6,060,051, 6,103,228, 6,291,426, 6,417,163 and 6,638,910. In these patents, the strain is referred to as AQ713, which is synonymous with QST713; WO2020102592: Bacillus thuringiensis strains NRRL B-67685, NRRL B-67687, and NRRL B-67688; WO2019135972: Bacillus megatherium having the deposit accession number NRRL B-67533 or NRRL B-67534; WO2019035881: Paenibacillus sp. NRRL B-50972, Paenibacillus sp. NRRL B-67129, Paenibacillus sp. NRRL B-67304, Paenibacillus sp. NRRL B-67615, Bacillus subtilis strain QST30002 deposited under accession no. NRRL B-50421, and Bacillus subtilis strain NRRL B-50455; WO2018081543: Bacillus psychrosaccharolyticus strain deposited under ATCC accession number PT A-123720 or PT A-124246; WO2017151742: Bacillus subtilis assigned the accession number NRRL B-21661; WO2016106063: Bacillus pumilus NRLL B-30087; WO2013152353: Bacillus sp. deposited as CNMC 1-1582; WO2013016361: Bacillus sp. strain SGI-015-F03 deposited as NRRL B-50760, Bacillus sp. strain SGI-015-H06 deposited as NRRL B-50761; WO2020181053: Paenibacillus sp. NRRL B-67721, Paenibacillus sp. NRRL B-67723, Paenibacillus sp. NRRL B-67724, Paenibacillus sp. NRRL B-50374; WO2020061140: Paenibacillus sp. NRRL B-67306.

The spores can according to the invention be derived from wild type or genetically modified microorganisms. Wild type microorganism samples preferably are recorded as type strains in culture collections. Genetic modification can be effected by random mutagenesis, for example NTG chemical mutagenesis, UV irradiation or transposon mutagenesis, or by directed mutagenesis, e.g. incorporation of heterologous plasmids or homologous recombination with heterologous nucleic acids and/or by site directed mutagenesis, e.g. using meganucleases, TALEN or CRISPR-type mutagenesis. For example, preferred methods of Bacillus and Paenibacillus mutagenesis are described in WO2017117395, incorporated herein in its entirety.

As described above, the composition preferably comprises spores according to the invention of one or more Paenibacillus species, more preferably of any of Paenibacillus alvei, Paenibacillus macerans, Paenibacillus nov. spec epiphyticus, Paenibacillus polymyxa, Paenibacillus polymyxa ssp. polymyxa, Paenibacillus polymyxa ssp. plantarum or Paenibacillus terrae, wherein the Paenibacillus species most preferably is a fusaricidin producing strain. Such Paenibacillus species have been extensively studied and mutagenized, e.g. to reduce formation of slime and correspondingly decrease viscosity in liquid phase fermentations. Thus, preferred Paenibacillus strains and methods of their manufacture are further described in any of WO2020181053, WO2019221988, WO2016154297, WO2017137351, WO2017137353 and WO2016020371.

As indicated above, the spore composition of the present invention preferably comprises one or more biopesticides, be it in spore form, adsorbed or attached thereto or in addition to the spores.

Such biopesticides preferably are chosen from:
L1) Microbial pesticides with fungicidal, bactericidal, viricidal and/or plant defense activator activity: Ampelomyces quisqualis, Aspergillus flavus, Aureobasidium pullulans, Bacillus altitudinis, Bacillus amyloliquefaciens, Bacillus licheniformis, Bacillus megaterium, Bacillus mojavensis, Bacillus mycoides, Bacillus pumilus, Bacillus simplex, Bacillus solisalsi, Bacillus subtilis, Bacillus subtilis var. amyloliquefaciens, Candida oleophila, Candida saitoana, Clavibacter michiganensis (bacteriophages), Coniothyrium minitans, Cryphonectria parasitica, Cryptococcus albidus, Dilophosphora alopecuri, Fusarium oxysporum, Clonostachys rosea f. catenulata (also named Gliocladium catenulatum), Gliocladium roseum, Lysobacter antibioticus, Lysobacter enzymogenes, Metschnikowia fructicola, Microdochium dimerum, Microsphaeropsis ochracea, Muscodor albus, Paenibacillus alvei, Paenibacillus epiphyticus, Paenibacillus polymyxa, Paenibacillus agglomerans, Pantoea vagans, Penicillium bilaiae, Phlebiopsis gigantea, Pseudomonas chlororaphis, Pseudomonas fluorescens, Pseudomonas putida, Pseudozyma flocculosa, Pichia anomala, Pythium oligandrum, Sphaerodes mycoparasitica, Streptomyces griseoviridis, Streptomyces lydicus, Streptomyces violaceusniger, Talaromyces flavus, Trichoderma asperellum, Trichoderma atroviride, Trichoderma asperelloides, Trichoderma fertile, Trichoderma gamsii, Trichoderma harmatum, Trichoderma harzianum, Trichoderma polysporum, Trichoderma stromaticum, Trichoderma virens, Trichoderma viride, Typhula phacorrhiza, Ulocladium oudemansii, Verticillium dahlia, zucchini yellow mosaic virus (avirulent strain);
L2) Biochemical pesticides with fungicidal, bactericidal, viricidal and/or plant defense activator activity: chitosan (hydrolysate), fusaricidins, paeniserines, paeniprolixines, harpin protein, laminarin, Menhaden fish oil, natamycin, Plum pox virus coat protein, potassium or sodium bicarbonate, Reynoutria sachalinensis extract, salicylic acid, tea tree oil (Melaleuca alternifolia extract);
L3) Microbial pesticides with insecticidal, acaricidal, molluscidal and/or nematicidal activity: Agrobacterium radiobacter, Bacillus cereus, Bacillus firmus, Bacillus subtilis, Bacillus licheniformis, Bacillus thuringiensis, Bacillus thuringiensis ssp. aizawai, Bacillus thuringiensis ssp. israelensis, Bacillus thuringiensis ssp. galleriae, Bacillus thuringiensis ssp. kurstaki, Bacillus thuringiensis ssp. tenebrionis, Beauveria bassiana, Beauveria brongniartii, Burkholderia rinojensis, Chromobacterium subtsugae, Cydia pomonella granulovirus (CpGV), Cryptophlebia leucotreta granulovirus (CrIeGV), Flavobacterium spp., Helicoverpa armigera nucleopolyhedrovirus (HearNPV), Heterorhabditis bacteriophora, Isaria fumosorosea, Lecanicillium longisporum, Lecanicillium muscarium, Metarhizium anisopliae, Metarhizium anisopliae var. anisopliae, Metarhizium anisopliae var. acridum, Nomuraea rileyi, Paecilomyces lilacinus, Paenibacillus popilliae, Pasteuria nishizawae, Pasteuria penetrans, Pasteuria ramosa, Pasteuria thornea, Pasteuria usgae, Phasmarhabditis hermaphrodita, Pseudomonas fluorescens, Spodoptera littoralis nucleopolyhedrovirus (SpliNPV), Steinernema carpocapsae, Steinernema feltiae, Steinernema kraussei, Steinernema riobrave, Streptomyces galbus, Streptomyces microflavus, Paecilomyces lilacinus;
L4) Biochemical pesticides with insecticidal, acaricidal, molluscidal, pheromone and/or nematicidal activity: L-carvone, citral, (E,Z)-7,9-dodecadien-1-yl acetate, ethyl formate, (E,Z)-2,4-ethyl decadienoate (pear ester), (Z,Z,E)-7,11,13-hexadecatrienal, heptyl butyrate, isopropyl myristate, lavanulyl senecioate, cis-jasmone, 2-methyl 1-butanol, methyl eugenol, methyl jasmonate, (E,Z)-2,13-octadecadien-1-ol, (E,Z)-2,13-octadecadien-1-ol acetate, (E,Z)-3,13-octadecadien-1-ol, R-1-octen-3-ol, pentatermanone, potassium silicate, sorbitol actanoate, (E,Z,Z)-3,8,11-tetradecatrienyl acetate, (Z,E) 9,12-tetradecadien-1-yl acetate, Z-7-tetradecen-2-one, Z-9-tetradecen-1-yl acetate, Z-11-tetradecenal, Z-11-tetradecen-1-ol, Acacia negra extract, extract of grapefruit seeds and pulp, Chenopodium ambrosioides extract, Catnip oil, Neem oil, Quillay extract, Tagetes oil;
L5) Microbial pesticides with plant stress reducing, plant growth regulator, plant growth promoting and/or yield enhancing activity: Azospirillum amazonense, Azospirillum brasilense, Azospirillum lipoferum, Azospirillum irakense, Azospirillum halopraeferens, Bradyrhizobium elkanii, Bradyrhizobium japonicum, Bradyrhizobium spp., Bradyrhizobium liaoningense, Bradyrhizobium lupini, Delftia acidovorans, Glomus intraradices, Mesorhizobium spp., Mesorhizobium ciceri, Rhizobium leguminosarum bv. phaseoli, Rhizobium leguminosarum bv. trifolii, Rhizobium leguminosarum bv. viciae, Rhizobium tropici, Sinorhizobium meliloti, Sinorhizobium medicae;
L6) Biochemical pesticides with plant stress reducing, plant growth regulator and/or plant yield enhancing activity: abscisic acid, aluminium silicate (kaolin), 3-decen-2-one, formononectin, genistein, hesperetin, homobrassinolide, humates, methyl jasmonate, cis-jasmone, lysophosphatidyl ethanlamine, naringenin, polymeric polyhydroxy acid, salicylic acid, Ascophyllum nodosum (Norwegian kelp, Brown kelp) extract and Ecklonia maxima (kelp) extract, zeolite (aluminosilicate), grape seed extract.

Exemplary compositions for agricultural uses comprising at least one Paenibacillus strain are described in WO2020064480, WO2019012379, WO2018202737, WO2017137351, WO2017137353, WO2017093163, WO2016202656, WO2016142456, WO2016128239, WO2016071164, WO2016059240, WO2016034353, WO2016020371, WO2015180983, WO2015180985, WO2015181035, WO2015180987, WO2015181008, WO2015180999, WO2015181009, WO2015177021, WO2015104698, WO2015091967, WO2015055752, WO2015055755, WO2015055757, WO2015011615, WO2015003908, WO2014202421, WO2014147528, WO2014095932, WO2014095994, WO2014086850, WO2014086851, WO2014086853, WO2014086854, WO2014086856, WO2014086848, WO2014076663, WO2014056780, WO2014053404, WO2014053405 and WO2014053398, WO2020131413, WO2020126980, WO2020092017, WO2020092022, WO2020065025, WO2020061140, WO2020056070, WO2020043650, WO2019222253, WO2019104173, WO2019094368, WO2019076891, WO2018026773, WO2018026774, WO2018026770, WO2017132330, WO2016018887, WO2016001125, WO2015004260, WO2014201326, WO2014201327, WO2014170364, WO2014152132, WO2014152115, WO2014127195, WO2014086752, WO2014083033, WO2013110591, WO2013110594 and WO2012140212. The present invention improves on the teaching of these publications by providing spores of the respective Paenibacillus strain in a particularly storage stable form and using methods that allow for a particularly efficient and fast production of such spore compositions.

It is particularly preferred that a composition according to the present invention comprises at least one fusaricidin, paeniserine or paeniprolixine, preferably at least two or more fusaricidins, more preferably 3 to 40, more preferably 2-10 fusaricidins which constitute at least 50 mol % of total fusaricidins of the composition, more preferably 2-10 fusaricidins which constitute at least 60 mol % of total fusaricidins of the composition, more preferably 2-10 fusaricidins which constitute at least 70 mol % of total fusaricidins of the composition, more preferably 2-10 fusaricidins which constitute at least 80 mol % of total fusaricidins of the composition. In each case it is particularly preferred that the one or more of the fusaricidins comprise any of fusaricidin A, B or D. Preferably the composition comprises, in addition to or instead of the at least one fusaricidin, surfactin and/or iturin. Such fusaricidins, surfactin and iturin are particularly effective biopesticides having bactericidal and/or fungicidal activity. Furthermore as shown herein the compositions of the present inventions allow for high yield production of such fusaricidins and their incorporation in agricultural products. Thus, the compositions of the present invention are particularly suitable for use as biopesticides and/or for use in anti-fungal and/or anti-bacterial plant health products.

The spores will typically be produced in a liquid phase fermentation and will be purified from the fermentation broth, for example by concentration. The fermentation broth or broth concentrate can be dried with or without the addition of carriers using conventional drying processes or methods such as spray drying, freeze drying, tray drying, fluidized-bed drying, drum drying, or evaporation. The resulting dry products may be further processed, such as by milling or granulation, to achieve a specific particle size or physical format. Carriers, described below, may also be added post-drying.

The spore composition according to the present invention preferably comprises at least one auxiliary selected from the group consisting of stabilisers (preferably: glycerol), extenders, solvents, surfactants, spontaneity promoters, solid carriers, liquid carriers, emulsifiers, dispersants, film forming agents, frost protectants, germinants, thickeners, plant growth regulators, inorganic phosphates, fertilizers, adjuvants, fatty acids and fibril, sugars, amino acids, microfibril or nanofibril structuring agents.

The carrier preferably has a sufficient shelf life, and preferably allows an easy dispersion or dissolution on a plant, plant part or in the volume of soil near the root system. Preferably the carrier has a good moisture absorption capacity, is easy to process and free of lump-forming materials, is near-sterile or easy to sterilize by autoclaving or by other methods (e.g., gamma-irradiation), and/or has good pH buffering capacity. For carriers that are used for seed coating, good adhesion to seeds is preferred.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e.g. toluene, paraffin, tetrahydronaphthalene, alkylated naphthalenes; alcohols, e.g. ethanol, propanol, butanol, benzylalcohol, cyclohexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, urea; products of vegetable origin, e.g. peat, cereal meal, tree bark meal, wood meal, nutshell meal, and mixtures thereof.

Suitable surfactants are surface active compounds, such as anionic, nonionic, cationic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants include alkali, alkaline earth or ammonium salts of sulfonates, sulfates, phosphates, carboxylates, and mixtures thereof. Examples of sulfonates are alkylarylsulfonates, diphenylsulfonates, alpha-olefin sulfonates, lignin sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkylnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants include alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. For example, ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinylpyrrolidone, vinylalcohols, or vinylacetate.

Said at least one nonionic surfactant preferably is at least one polyalkyleneoxide PAO. Polyalkyleneoxides PAO comprise blocks of polyethylene oxide (PEO) at the terminal positions, whereas blocks of polyalkylene oxides different from ethylene oxide like polypropylene oxide (PPO), polybutylene oxide (PBO) and poly-THF (pTHF) are comprised in central positions. Preferred polyalkyleneoxides PAO have the structure PEO-PPO-PEO, PPO-PEO-PPO, PEO-PBO-PEO or PEO-pTHF-PEO. Suitable polyalkyleneoxides PAO normally comprise a number average of 1.1 to 100 alkyleneoxide units, preferably 5 to 50 units.

Suitable cationic surfactants include quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a negligible or even no pesticidal activity themselves, and which improve the biological performance of the spores, the compounds attached thereto or produced by germinating cells on the target. Examples are surfactants, mineral or vegetable oils, and other auxiliaries. Further examples are listed by Knowles, Adjuvants and additives, Agrow Reports DS256, T&F Informa UK, 2006, chapter 5.

Compositions according to the invention preferably comprise 0.01 to 2 wt % of an organic or inorganic thickener. Suitable thickeners include polysaccharides (e.g. xanthan gum, carboxymethylcellulose), inorganic clays (organically modified or unmodified), polycarboxylates, and silicates.

Suitable thickeners are polysaccharides (e.g. xanthan gum, carboxymethylcellulose), anorganic clays (organically modified or unmodified), polycarboxylates, and silicates. A preferred thickener in a composition of the present invention is xanthan gum. Preferably xanthan gum is comprised in compositions according to the invention in an amount of 0.01 to 0.4 wt %, preferably 0.05 to 0.15 wt %, based on the formulation.

Compositions according to the invention preferably comprise a magnesium aluminum silicate (for example montmorillonite and/or saponite), bentonites, attapulgites or silica as a thickener. The content of magnesium aluminum silicate (e.g. montmorillonite and saponite), bentonite, attapulgite or silica is preferably is of 0.1 to 2 wt % of the total composition, preferably 0.5 to 1.5 wt %.

Suitable anti-foaming agents are silicones, long chain alcohols, and salts of fatty acids. Preferably compositions according to the invention contain 0.01 to 1.0 wt % of an anti-foaming agent, for example of a silicone anti-foaming agent.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants). Suitable bactericides are bronopol and isothiazolinone derivatives such as alkyliso-thiazolinones and benzisothiazolinones. Suitable anti-freezing agents are ethylene glycol, propylene glycol, urea and glycerin. Suitable anti-foaming agents are silicones, long chain alcohols, and salts of fatty acids. Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants). Suitable tackifiers or binders are polyvinylpyrrolidons, polyvinylacetates, polyvinyl alcohols, polyacrylates, biological or synthetic waxes, and cellulose ethers.

Suitable fibril, microfibril and nanofibril auxiliaries and their incorporation into agricultural compositions are described for example in WO2019035881.

Preferably the composition is a plant pest control composition and/or prevents, limits or reduces a phytopathogenic fungal or bacterial disease and/or improves or promotes the health of a plant and/or increases or promotes yield of plants when applied to such plant, a part or propagation material thereof or to the substrate where the plants are to grow. As described herein, it is an advantage of the spore compositions of the present invention that spores can be incorporated in the composition which have a short lag phase duration in subsequent fermentation and a late end of log phase growth. The spores thus can rapidly germinate after spreading on plants, plant parts or plant growth substrate, e.g. soil, thereby exerting their plant beneficial properties, e.g. reduction of pathogenic microorganisms or making nutrients available to the plants or plant parts. In particular compositions of the present invention can be used, for example, to promote significantly improved transport and dispersal of beneficial bacteria and other agricultural payloads to rapidly growing plant roots.

The composition preferably comprises at least said spores in a concentration of at least 10\hat{ }4 colony forming units (cfu) per ml of the total composition, more preferably 10\hat{ }4-10\hat{ }17 cfu/ml, more preferably 10\hat{ }7-10\hat{ }13 cfu/ml. To exert a more noticeable or fast effect after application on a field or a patient or animal in need, it is particularly preferred that a composition of the present invention comprises at least 10\hat{ }6 cfu/ml of spores, more preferably 10\hat{ }7 to 10\hat{ }17 cfu/ml, more preferably 10\hat{ }8 to 10\hat{ }15 cfu/ml.

Furthermore a high spore concentration is advantageous in biotechnological cultivation processes, in particular for maintaining a master or working “cell” bank. Use of working cell banks containing or consisting of spores instead of pure viable cells is known to significantly increase storage stability of the seed and thus improves reproducibility of fermentation processes. In such cell banks it is mandatory that the stored microorganism material remains viable for extended periods of more than 1 year, preferably 1-5 years, without significant loss of germination and outgrowth activity. As described herein it is a particular advantage of the present invention to provide such compositions suitable for long-term storage under normal storage conditions. It is a further advantage that the compositions of the present invention do not suffer a significant reduction in germination frequency and speed even after such long storage. This was particularly surprising in view of the low content of dipicolinic acid in the spores of the present invention compared to compositions comprising a higher fraction of late formed spores.

A preferred master or working cell bank sample according to the present invention thus is a composition according to the present invention, wherein the composition comprises a cryoprotectant, preferably glycerol, in a sufficient quantity for cryoprotection. Cryoprotection is advised for storages at −180° C. but also at higher storage temperatures like −80° C., −20° C. up to 0° C. In addition, dried spores, e.g. obtained from freeze drying of a at least partially sporulated microbial culture can be used as working cell bank. Such compositions advantageously exhibit a good storage stability also at temperatures below 0° C., in particular −180° C. to −20° C., without requiring addition of cryoprotectants, e.g. glycerol. However, as the spores in the present composition already exhibit a surprisingly strong storage stability despite their comparatively low dipicolinic acid content, it is an advantage of the present invention that the amount of cryoprotectant can be reduced compared to standard cell bank sample compositions as described e.g. in F. S. (1995) Freeze-Drying and Cryopreservation of Bacteria. In: Day J. G., Pennington M. W. (eds) Cryopreservation and Freeze-Drying Protocols. Methods in Molecular Biology™, vol 38. Humana Press, Totowa, NJ. https://doi.org/10.1385/0-89603-296-5:21

The composition of the present invention preferably comprises added dipicolinic acid, preferably to a final content of 4×10\hat{ }-6 to 4×10\hat{ }-5 μmol/spore, more preferably 5×10\hat{ }-6 to 2×10\hat{ }-5 μmol/spore, more preferably 7×10\hat{ }-6 to 1×10\hat{ }-5 μmol/spore. Addition of dipicolinic acid to achieve the aforementioned concentrations further improves stability, i.e. germination frequency and speed, of the spores particularly when the composition has a low water content, e.g. when the composition is in powder or granule form, or when the composition is intended for storage at elevated temperatures, e.g. 4-45° C.

As described above the composition can comprise viable and/or non-viable cells. Preferably at least a fraction of the spores comprises on their surface a protein comprising a payload domain, said protein also comprising a targeting domain for delivery of the payload domain to the surface of said spores. Examples of preferred proteins, spores and methods of their production are described in WO2020232316 and WO2019099635.

The composition of the present invention can be readily used as a product on its own. However, the composition of the present invention can also be a part of a kit. This is particularly useful in situations where an application together with or in timely proximity to harmful chemicals or treatments is desired, such that the composition of the present invention can be kept separate from the potentially harmful further kit components.

In particular, the composition of the present invention preferably is used as or incorporated in a paint, coat or impregnation composition for the treatment of mineral surfaces and/or for the preparation of a cement. As described above, Clostridia spores comprised in a composition of the present invention are able to germinate even after long periods of time and provide metabolic calcification processes to improve healing of cracks.

Furthermore the invention provides a food or feed product comprising a composition according to the present invention. preferably a probiotic or prebiotic food or feed product. As described above various endospores of aerobic and anaerobic microorganisms are valuable probiotic agents; they may also contain prebiotic substances. Thus, the invention advantageously provides pro- and/or prebiotic food and feed compositions in desired ratios of early to late spore communities to achieve, in a plannable way, the benefits conferred by those spore communities. In such compositions the spores will be selected from probiotic or prebiotic species. Such species, when administered in adequate amounts, confer a health benefit on the host. Preferred species are Bacillus amyloliquefaciens, Bacillus aquimaris, Bacillus aryabhattai, Bacillus cereus, Bacillus clausii, Bacillus coagulans, Bacillus flexus, bacillus fusiformis, Bacillus indicus, Bacillus licheniformis, Bacillis megatherium, Bacillus polyfermenticus, Bacillus pumilus, Bacillus subtilis, Bacillus thuringiensis, Bacillus vireti, Clostridium butyricum, Clostridium cellulosi, Clostridium leptum, Clostridium sporosphaeroides, Faecalibacterium prausnitzii Paenibacillus ehimensis, Paenibacillus elgii, Paenibacillus pabuli and Paenibacillus polymyxa.

The invention furthermore provides a plant protection product, comprising a plant cultivation substrate coated or infused with a composition according to the present invention or obtainable or obtained by a method according to the present invention. Such products realize the advantages conferred by a composition according to the present invention. In particular, such products can provide biopesticidal spores and compounds attached to spores, and preferably said spores germinate fast and reliably as described herein. Thus, a plant cultivation substrate according to the present invention particularly facilitates the germination and outgrowth of plant health beneficial microorganisms from said spores. Preferably the plant protection product improves one or more plant health indicators and/or reduces pathogen pressure due to said germinated microorganisms compared to an untreated plant cultivation substrate.

The beneficial effect of the present composition is preferred observed in one or more of the following plant health indicators: early and better germination, less seeds needed without compromising the number of fruit-bearing plants, earlier or more durable emergence, improved root formation, increased root density, increased root length, improved root size maintenance, improved root effectiveness, improved nutrient uptake, preferably of nitrogen and/or phosphorus, increased shoot growth, enhanced plant vigor, increased plant stand, increase in plant height, bigger leaf blade, less dead basal leaves, tillering increase, stronger tillers, more productive tillers, increased tolerance against stress (e.g. against drought, heat, salt, UV, water, cold), reduced need for fertilizers, pesticides and/or water, reduced ethylene production and/or reduced ethylene reception, increased photosynthetic activity, greener leaf color, improved pigment content, earlier flowering, early grain maturity, increased crop yields, increased protein content in fruit or seed, increased oil content in fruit or seed and increased starch content in fruit or seed. In view of the biopesticidal actin of the preferred composition of the present invention, most preferably a composition wherein the spores comprise or consist of those of genus Paenibacillus, even more preferably spores of Paenibacillus polymyxa, Paenibacillus polymyxa polymyxa, Paenibacillus polymyxa plantarum and/or Paenibacillus terrae, such composition of the present invention can reduce the need for chemical pesticide treatments of plants, plant parts or plant growth substrates. Agricultural compositions of the present invention thus advantageously improve safety of plant products by helping to reducing the need for exposure to chemical pesticides.

The invention also provides a plant, plant part or plant propagation material, wherein the material comprises, on its surface or infused therein, a composition according to the present invention or obtainable or obtained by a method according to the present invention. A method of seed infusion is described, for example, in WO2020214843. As described herein, the spores in a composition of the present invention in particular have a reliable and fast germination speed. Thus, the spores support rapid colonization of plant material including seed, root, leaves and stalk, thereby promoting one or more of the plant health indicators by exerting the beneficial effects imparted by the spores and/or germinated microorganisms.

Furthermore, the invention provides plantation, preferably a field or a greenhouse bed, comprising a plant, plant part or plant propagation material or a plant cultivation substrate as described above. As described above it is an advantage of the present invention that spores of the composition of the present invention and/or corresponding germinated microorganisms exert biopesticidal effects. Thus, a plantation treated with a composition or product of the present invention advantageously prevents, delays, limits or reduces the emission of phytopathogenic fungal or bacterial material from a plantation, preferably due to increased and/or accelerated outgrowth of microorganisms from the spores of the composition. Application of a composition or product of the present invention on a plant cultivation area, e.g. on plants, plant material and/or soil thereof, not only helps to reduce pests on the area. As the pest preferably does not multiply as fast on the area as on an untreated area, less pests will escape from said area to infest neighbouring areas. Thus, application of the product or composition of the present invention advantageously not only reduces the number of pesticide treatments on site but can also provide such savings to adjacent areas.

The invention also provides a cleaning or cosmetic product comprising a composition according to the invention. As described above, spores can advantageously improve the properties of cleaning products, for example of skin cleaning products, hair cleaning products, laundry products, dishwashing products, pipe degreasers, allergen removal products, more preferably a cosmetic foundation, lipstick, cleanser, exfoliant, blush, eyeliner, eye shadow, lotion, cream, shampoo, toothpaste, tooth gel, mouth rinse, dental floss, tape or toothpick. As described herein, in such products it is advantageous to define a ratio of early to late spore communities such as to achieve a desired degree of fast spore action and longer lasting effects of late germinating spores. Preferably the cleaning product comprises a detergent and at least one component selected from surfactants, builders, and hydrotropes is present in an amount effective in cleaning performance or effective in maintaining the physical characteristics of the detergent. Examples of such components are described e.g. in “complete Technology Book on Detergents with Formulations (Detergent Cake, Dishwashing Detergents, Liquid & Paste Detergents, Enzyme Detergents, Cleaning Powder & Spray Dried Washing Powder)”, Engineers India Research Institute (EIRI), 6th edition (2015), or in “Detergent Formulations Encyclopedia”, Solverchem Publications,

Correspondingly the invention also provides a method of producing a composition comprising spores of a prokaryotic microorganism, comprising the steps of
1) fermenting the microorganism in a medium conductive to sporulation,
2) purifying the spores to obtain the composition.

As described above, the method provides a fast and reliable way to produce compositions of the present invention. It is a particular advantage that the method of the present invention can be performed using standard industrial equipment and fermentation routines which either are already established for the microorganisms in question or can be adapted from related industrially relevant strains.

Purification, also called harvesting, is the last step of a batch liquid phase fermentation. The goal of purification is generally to remove or reduce fermentation media components which would destabilize endospores during storage in a composition of the present invention. Preferred purification steps are described herein; preferably purification comprises concentrating of spores and preferably comprises a step of desiccation, lyophilization, homogenization, extraction, tangential flow filtration, depth filtration, centrifugation or sedimentation. The resulting concentrated spore preparation, preferably a preparation depleted in viable cells, even more preferably a cell-free preparation, may then be dried and/or formulated with further components as described herein.

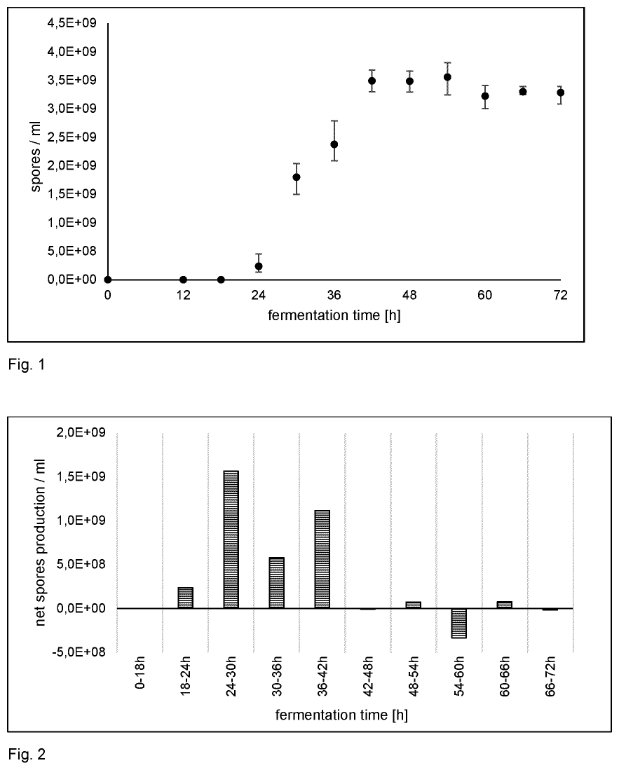

Preferably purification is performed latest when 85% of the maximum viable spore concentration obtainable in the fermentation step 1) is reached, more preferably purification is performed when a concentration in the range of 1-75% is reached, more preferably when a concentration in the range of 10-75% is reached, more preferably when a concentration in the range of 20-70% is reached, more preferably when a concentration in the range of 30-68% is reached. To this end, first a calibration fermentation is performed in the chosen medium and under the chosen fermentation conditions. The calibration fermentation is performed until no further increase in biomass is observed after log phase, preferably until the biomass increases by less than 1% per 6 hours. In the fermentation according to FIG. 1, the spore concentration determined at 48 h is thus taken as maximum spore concentration. By harvesting the spores at the indicated level of maturity, a composition of the present invention comprising a high share of spores of the early spore community can be obtained. Thus, purification preferably is performed such that said purified spores form colonies when plated on a medium suitable for colony formation, and wherein of all such colonies formed within 72 h for aerobic cultures and 96 h for anaerobic cultures after plating at least 40% have formed within 48 h, more preferably 40-90%, more preferably at least 50%, more preferably 50-90%, more preferably at least 60%, more preferably 60-90%, more preferably at least 70%, more preferably 70-90%, and/or such that said purified at least 40% of spores are obtainable or obtained from a fermentation harvested during a first spore formation phase, more preferably at least 50%, more preferably at least 55%, more preferably at least 60%, more preferably at least 70%, more preferably at least 80%.

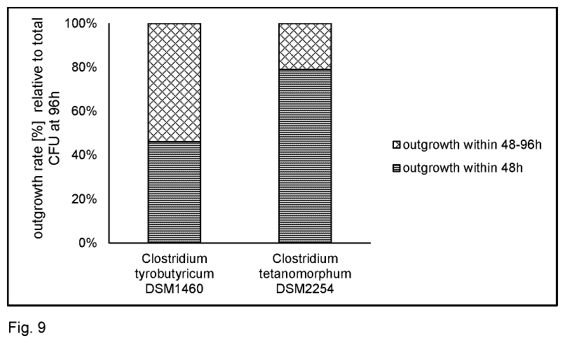

Another preferred method of determining a suitable time for purification from liquid phase fermentation is when the mean content of dipicolinic acid per spore is at most 80% of the mean content of dipicolinic acid of spores produced when reaching maximum spore concentration in the fermentation step 1), herein also called plateau phase, more preferably the mean content of dipicolinic acid is in the range of 20-80%, even more preferably in the range of 22-70%, even more preferably in the range of 30-65%. As described in the examples, a calibration fermentation is performed first and both dipicolinic acid content of spores and viable spore concentration are measured. Then, the concentration of dipicolinic acid per viable spores is calculated. As shown for example in FIG. 9, the ratio of dipicolinic acid per spore levels off; when ratio no longer increases or at least does no longer increase by more than 3% per 6 h, the ratio is set to have reached 100% and the concentration of dipicolinic acid is set to be maximal. All further percentages can then be calculated on these values. As indicated above, by harvesting the spores at the indicated dipicolinic acid content, a composition of the present invention comprising a high share of spores of the early spore community can be obtained.

As described next to the examples' section, the invention furthermore provides methods for producing a composition comprising a high fraction of a late spore community, and also provides uses and advantages thereof.

Preferably the dipicolinic acid content of the composition is further increased after purification, for example by addition of externally produced dipicolinic acid. It has been described by Daniel et al., J. Mol. Biol. 1993, 468-483 that addition of dipicolinic acid to spores can further improve spore storage stability.

As indicated above, the purification step 2) preferably results in a suppression or reduction of spore germination in the composition as such. This leads to an advantageous further improvement of storage stability and viability of the spores in the composition of the present invention at storage temperatures of −20° C. to 45° C. Thus, the purification step preferably comprises a step of desiccation, lyophilization, homogenization, extraction, filtration, centrifugation, sedimentation, or concentration of spores, and/or comprises adjusting the water content of the composition to approximately 1-8% (w/w), preferably 3-5% by weight of the composition for dry compositions, and 10-98% by weight of the composition for liquid or pasty compositions, and/or comprises adjusting the soluble carbon source content of the composition to at most 50% by weight of the composition compared to its content at the time of spore harvest, more preferably 5-30% by weight of the composition. Such methods of downstream processing are generally known to the skilled person, they can be performed using standard industry equipment and using minimal adaptation of methods known in the art. It is thus a particular advantage of the present invention that the compositions of the present invention can easily be produced at low costs.

Furthermore the method preferably also comprises addition of at least one pest control agent preferably selected from the group consisting of
i) one or more microbial pesticides with fungicidal, bactericidal, viricidal and/or plant defense activator activity,
ii) one or more biochemical pesticides with fungicidal, bactericidal, viricidal and/or plant defense activator activity,
iii) one or more microbial pesticides with insecticidal, acaricidal, molluscidal and/or nematicidal activity,
iv) one or more biochemical pesticides with insecticidal, acaricidal, molluscidal, pheromone and/or nematicidal activity,
v) one or more fungicide selected from respiration inhibitors, sterol biosynthesis inhibitors, nucleic acid synthesis inhibitors, inhibitors of cell division and cytoskeleton formation or function, inhibitors of amino acid and protein synthesis, signal transduction inhibitors, lipid and membrane synthesis inhibitors, inhibitors with multi site action, cell wall synthesis inhibitors, plant defence inducers and fungicides with unknown mode of action. The advantages of such additional pesticides and treatment agents have been described above.

It is also preferred that the method further comprises addition of at least one fusaricidin, preferably at least two or more fusaricidins, paeniserine or paeniproxilin, more preferably 3 to 40 fusaricidins, more preferably 2-10 fusaricidins which constitute at least 50 mol % of total fusaricidins of the composition, more preferably 2-10 fusaricidins which constitute at least 60 mol % of total fusaricidins of the composition, more preferably 2-10 fusaricidins which constitute at least 70 mol % of total fusaricidins of the composition, more preferably 2-10 fusaricidins which constitute at least 80 mol % of total fusaricidins of the composition. In each case it is particularly preferred that the one or more of the fusaricidins comprise any of fusaricidin A, B or D. Preferably the method comprises, in addition to or instead of the at least one fusaricidin addition, added surfactin and/or iturin and/or further comprises addition of at least one auxiliary selected from the group consisting of stabilisers (preferably: glycerol), extenders, solvents, surfactants, spontaneity promoters, solid carriers, liquid carriers, emulsifiers, dispersants, film forming agents, frost protectants, thickeners, plant growth regulators, inorganic phosphates, fertilizers, adjuvants, fatty acids and fibril, microfibril or nanofibril structuring agents. Again, the correspondingly obtainable advantages have been described above.

The invention also provides a fermentation method, comprising the step of inoculating a fermenter comprising a suitable fermentation medium with a composition of the present invention or a composition obtainable or obtained by a method according to the invention. As described above, it is a particular advantage that the spores in the composition according to the present invention show a fast germination behaviour, even after storage. Thus, the composition of the present invention is advantageously suitable for the preparation of rapidly usable master or working cell banks.

Correspondingly the invention provides a method for controlling, in a fermentation of spore-forming prokaryotic microorganisms, the duration of a lag phase and/or the time until reaching the end of log phase, comprising inoculating a suitable fermentation medium with a composition of the invention or a composition obtainable or obtained by a method according to any the invention, and fermenting the inoculated medium, wherein for shorter duration of the lag phase and/or faster end of log phase a composition is used having a higher percentage of spores harvested in a first spore formation phase, and for longer duration of lag phase or later end of log phase a composition is used having a higher percentage of spores harvested in a second spore formation phase. Thus, when conducting a batch fermentation it is expedient that the skilled person purifies spores from said fermentation reaction at a time which provides the desired content of rapidly germinating spores. In particular the invention improves planning and adjustment of harvesting times of industrial batch fermentations. As the time for completion of the fermentation batch can be reliably predicted according to the contents of the composition of the present invention. Thus, the time for reaching a predefined fermentation stage can be adjusted by choosing the appropriate composition of the present invention for inoculation. As described and preferred herein, a high content of spores of a first spore formation phase is obtainable latest when 85% of the maximum spore concentration obtainable in the fermentation step 1) is reached, more preferably when a concentration in the range of 1-75% is reached, more preferably when a concentration in the range of 10-75% is reached, more preferably when a concentration in the range of 20-70% is reached, more preferably when a concentration in the range of 30-68% is reached; alternatively, a high content of spores of a first spore formation phase are obtainable when the mean content of dipicolinic acid per spore is at most 80% of the mean content of dipicolinic acid of spores produced when reaching maximum spore concentration in the fermentation step 1), more preferably the mean content of dipicolinic acid is in the range of 20-80%, even more preferably in the range of 22-70%, even more preferably in the range of 30-65%.

Preferably the method for controlling, in a fermentation of spore-forming prokaryotic microorganisms, the duration of a lag phase and/or the time until reaching the end of log phase, is a computer implemented method, comprising the steps of (1) obtaining a target growth signal and (2) adjusting the contents of an inoculating composition such that for shorter duration of the lag phase and/or faster end of log phase a composition is used having a higher percentage of spores harvested in a first spore formation phase, and for longer duration of lag phase or later end of log phase a composition is used having a higher percentage of spores harvested in a second spore formation phase. In particular, a fermentation reactor is preferably connected to an inoculation sample storage comprising compositions of the present invention, i.e. a collection of working cell bank samples. For each composition the content of the early spore community is recorded as described herein, preferably by sample plating and recording the percentage of colonies formed within the first observation period of 48 h of a 72 h for aerobic cultures and 96 h for anaerobic cultures, or also preferably by recording the stage of the fermentation at the time when the spores were purified for the composition, e.g. the percentage of spores are obtained from a fermentation harvested during a first spore formation phase, the mean content of dipicolinic acid per spore or the percentage of the maximum spore concentration achievable in such fermentation. The inoculation sample storage comprises a computer equipped for performing the above computer-implemented method. Upon receiving a timing signal indicating the desired lag phase duration or end of log phase, the computer determines which working cell bank sample fits best to the timing signal. Preferably, the computer emits a timing prediction indicative for the expected duration of lag phase or until end of log phase so that the user can reconsider the timing signal and possibly correct the timing signal. When the definitive timing signal is received by the computer and the appropriate working cell bank sample is chosen, the computer (1) emits an identifier of the chosen sample to allow retrieval of the sample from the working cell bank sample collection by the user for fermenter inoculation, and/or (2) automatically performs retrieval of the chosen sample from the working cell bank sample collection and hands over the retrieved sample to the user for fermenter inoculation, or (3) automatically performs dosing of the chosen sample from the working cell bank sample collection to the fermenter, or (4) automatically mixes a new working cell bank sample by adjusting the proportion of early and late spore communities by drawing from an early spore community enriched and from a late spore community enriched stock, respectively.

The invention also provides a method of promoting spore germination and/or vegetative growth of a spore-forming prokaryotic microorganism, comprising providing spores harvested during a first spore formation phase in a method of the present invention, wherein preferably inorganic phosphate is provided together or sequentially with the spores. The inorganic phosphate is preferably chosen from phosphoric acid, polyphosphoric acid, phosphorous acid and/or a salt of H2PO4\hat{ }(−), H2PO3\hat{ }(−), HPO4\hat{ }(2−) or PO4\hat{ }(3−). Preferably the inorganic phosphate is selected from the group consisting of monoammonium phosphate, diammonium phosphate, monopotassium phosphate, dipotassium phosphate, ammonium polyphosphate, calcium phosphate, monobasic calcium phosphate, bibasic calcium phosphate, magnesium phosphate, zinc phosphate, manganese phosphate, iron phosphate, potassium phosphite, copper phosphate, NPK fertilizer, rock phosphate and combinations thereof. As described for example in WO2018140542, application of 0.2 to 2.7 mg/ml of inorganic phosphate, preferably calcium phosphate, on a plant part, seed or the growth substrate for a plant—preferably soil—promotes spore germination and/or vegetative growth of Bacillus or Paenibacillus strains.

Furthermore, the invention provides a use of a composition of the present invention or obtainable or obtained by a method according to the invention
a) for inoculating a fermentation, or
b) for pest control and/or for preventing, delaying, limiting or reducing the intensity of a phytopathogenic fungal or bacterial disease and/or for improving the health of a plant and/or for increasing yield of plants and/of for preventing, delaying, limiting or reducing the emission of phytopathogenic fungal or bacterial material from a plant cultivation area.

As described above, such use realizes the advantages conferred by the composition or production method of the present invention. In particular, by preventing, delaying, limiting or reducing the intensity of phytopathogenic fungal or bacterial infestation, plant health is improved which, in turn, can lead to one or more advantageous effects: early and better germination, earlier or more durable emergence, increased crop yields, increased protein content, increased oil content, increased starch content, more developed root system, improved root growth, improved root size maintenance, improved root effectiveness, increased tolerance against stress (e.g., against drought, heat, salt, UV, water, cold), reduced ethylene production and/or reduced ethylene reception, tillering increase, increase in plant height, bigger leaf blade, less dead basal leaves, stronger tillers, greener leaf color, pigment content, increased photosynthetic activity, reduced need for fertilizers, pesticides and/or water, less seeds needed, more productive tillers, earlier flowering, early grain maturity, less plant verse (lodging), increased shoot growth, enhanced plant vigor and increased plant stand.

According to the invention provided is also a method of protecting a plant or part thereof in need of protection from pest damage, comprising contacting the pest, plant, a part or propagation material thereof or to the substrate where the plants are to grow with an effective amount of a composition according to the invention or obtainable or obtained by a method according to the invention, preferably before or after planting, before or after emergence, or preferably as particulates, a powder, suspension or solution. Preferably the composition is applied at about 1×10 to about 1×10 colony forming units (cfu) of the spores, preferably the Bacillus or Paenibacillus spores and most preferably of the Paenibacillus spores, per hectare or at about 0.5 kg to about 5 kg composition solids per hectare.

Furthermore provided by the invention is a method of delivering a protein payload to a plant, plant part, seed or growth substrate, comprising applying a composition according to the invention or obtainable or obtained by a method according to the invention to the plant, plant part, seed or substrate, wherein the spores are those of a microorganism expressing a protein comprising a payload domain and a targeting domain for delivery of the payload domain to the surface of said spores. As described above, suitable proteins for target domain delivery and methods for genetic manipulation of Paenibacillus strains are described for example in WO2020232316 and WO2019099635.

The invention in particular provides a use or method as described herein, wherein
i) the fungal disease is selected from white blister, downy mildews, powdery mildews, clubroot, sclerotinia rot, fusarium wilts and rots, botrytis rots, anthracnose, rhizoctonia rots, damping-off, cavity spot, tuber diseases, rusts, black root rot, target spot, aphanomyces root rot, ascochyta collar rot, gummy stem blight, alternaria leaf spot, black leg, ring spot, late blight, cercospora, leaf blight, septoria spot, leaf blight, or a combination thereof, and/or
ii) the fungal disease is caused or aggravated by a microorganism selected from the taxonomic ranks:
   class Sordariomycetes, more preferably of order Hypocreales, more preferably of family Nectriaceae, more preferably of genus Fusarium;
   class Sordariomycetes, more preferably of order Glomerellales, more preferably of family Glomerellaceae, more preferably of genus Colletotrichum;
   class Leotinomycetes, more preferably of order Helotiales, more preferably of family Sclerotiniaceae, more preferably of genus Botrytis;
   class Dothideomycetes, more preferably of order Pleosporales, more preferably of family Pleosporaceae, more preferably of genus Alternaria;
   class Dothideomycetes, more preferably of order Pleosporales, more preferably of family Phaeosphaeriaceae, more preferably of genus Phaeosphaeria;
   class Dothideomycetes, more preferably of order Botryosphaeriales, more preferably of family Botryosphaeriaceae, more preferably of genus Macrophomina;
   class Dothideomycetes, more preferably of order Capnodiales, more preferably of family Mycosphaerellaceae, more preferably of genus Zymoseptoria;
   class Agraricomycetes, more preferably of order Cantharellales, more preferably of family Ceratobasidiaceae, more preferably of genus Rhizoctonia or Thanatephorus;
   class Pucciniomycetes, more preferably of order Pucciniales, more preferably of family Pucciniaceae, more preferably of genus Uromyces or Puccinia;
   class Ustilaginomycetes, more preferably of order Ustilaginales, more preferably of family Ustilaginaceae, more preferably of genus Ustilago;
   class Oomycota, more preferably of order Pythiales, more preferably of family Pythiaceae, more preferably of genus Pythium;
   class Oomycota, more preferably of order Peronosporales, more preferably of family Peronosporaceae, more preferably of genus Phytophthora, Plasmopara or Pseudoperonospora.

Such fungal pests are responsible for widespread crop damage and/or yield decrease. It is particularly advantageous the composition of the present invention are suitable and adapted for preventing, delaying, limiting or reducing the intensity of infections by phytopathogenic fungi as listed above. In such uses or methods, the spores are preferably spores of genus Paenibacillus, more preferably Paenibacillus koreensis, Paenibacillus rhizosphaerae, Paenibacillus polymyxa, Paenibacillus amylolyticus, Paenibacillus terrae, Paenibacillus polymyxa polymyxa, Paenibacillus polymyxa plantarum, Paenibacillus nov. spec epiphyticus, Paenibacillus terrae, Paenibacillus macerans, Paenibacillus alvei, more preferred Paenibacillus polymyxa, Paenibacillus polymyxa polymyxa, Paenibacillus polymyxa plantarum, Paenibacillus nov. spec epiphyticus, Paenibacillus terrae, Paenibacillus macerans, Paenibacillus alvei, even more preferred Paenibacillus polymyxa, Paenibacillus polymyxa polymyxa, Paenibacillus polymyxa plantarum and Paenibacillus terrae and most preferably Paenibacillus polymyxa or Paenibacillus terrae.

As announced above the invention, in another aspect, also provides a method of producing a composition comprising spores of a prokaryotic microorganism, comprising the steps of
1) fermenting the microorganism in a liquid medium conductive to sporulation until the biomass increases by less than 1% of cells/4 h,
2) spiking the fermentation medium with nutrients to cause germination of spores in the medium, and
3) purifying late spores from the medium, wherein purification is performed
a) after reduction of spore concentration in the medium by 10%, more preferably by 20%, more preferably by 30%, more preferably by 40%, and/or
b) after increase of cell number in the medium by 2%, more preferably by 5%, more preferably by 10%,
   and wherein purification comprises a step of inactivating viable cells and/or spores in the process of formation, preferably by UV treatment and/or, more preferably, by heat treatment.

As described above a fully sporulated liquid phase fermentation will contain both an early spore community and a late spore community. Selectively enriching late community spores is not feasible by separation methods, as early and late spores are phenotypically nearly indistinguishable. However, due to the propensity of early spore communities to germinate rapidly, such early spores can be brought to germination and be inactivated before the majority of late spores have germinated. Thus, the invention provides a reliable, fast and uncomplicated method for providing spore compositions enriched in late community spores. It is an advantage of such compositions that the spores are very durable and can sporulate slowly but steadily over a long period of time. In an agricultural, cleaning or probiotic product, such compositions are thus advantageous to prolong the effects obtainable by early spore compositions even after such the spores of such compositions have germinated and viable cells have eventually been lost. Furthermore, such compositions depleted in spores of the early spore community and enriched in late spores are advantageous to deliberately be mixed with compositions enriched in early spore communities, e.g. for fermenter inoculation as described above.

Preferably the composition of the present invention comprises spores of two species, wherein the spores of one species are enriched in early germinating spores and the spores of the other species are enriched in late germinating spores. In more detail, the invention provides a composition comprising purified spores of at least two prokaryotic microorganisms, wherein
i) for the first species
a) said spores form colonies when plated on a medium suitable for colony formation, and wherein of all such colonies formed within 72 h for aerobic cultures and 96 h for anaerobic cultures after plating at least 40% are formed within 48 h, more preferably 40-90%, more preferably at least 50%, more preferably 50-90%, more preferably at least 60%, more preferably 60-90%, more preferably at least 70%, more preferably 70-90%, and/or
b) at least 40% of spores are obtainable or obtained from a fermentation harvested during a first spore formation phase, more preferably at least 50%, more preferably at least 55%, more preferably at least 60%, more preferably at least 70%, more preferably at least 80% and/or
c) the mean content of dipicolinic acid per spore is at most 80% of the mean content of dipicolinic acid of spores fermented in a suitable medium until plateau phase, more preferably 20-80%, even more preferably 22-70%, even more preferably 30-65%, and
ii) for the second species
a) said spores form colonies when plated on a medium suitable for colony formation, and wherein of all such colonies formed within 72 h for aerobic cultures and 96 h for anaerobic cultures after plating at least 30% are formed after 48 h, more preferably 40-90%, more preferably at least 50%, more preferably 50-90%, more preferably at least 60%, more preferably 60-90%, more preferably at least 70%, more preferably 70-90%, and/or
b) at least 40% of spores are obtainable or obtained from a fermentation harvested during a second spore formation phase, more preferably at least 50%, more preferably at least 55%, more preferably at least 60%, more preferably at least 70%, more preferably at least 80% and/or
c) the mean content of dipicolinic acid per spore is at least 70% of the mean content of dipicolinic acid of spores fermented in a suitable medium until plateau phase, more preferably 80-100%, even more preferably 85-100%, even more preferably 90-100%.

Such compositions beneficially allow, during use of the composition, to have the spores of the first species germinate and grow fast after application of the composition, e.g. to a plant, plant part or plant growth substrate, whereas the second species will germinate later and over a longer period, thereby providing the corresponding beneficial effects consistently over a longer period of time.

The invention is hereinafter further illustrated by way of the following non-limiting examples.

### EXAMPLES

### Example 1: Spore Formation of Paenibacillus STRAIN 32 in 12 L Scale Fermentation

For monitoring spore formation of Paenibacillus and Bacillus strains over the course of a fermentation, 12 L scale fermentations were carried. From this, as an example, the number of spores/ml in such a fermentation using Paenibacillus strain STRAIN 32 is depicted in FIG. 1.

Strain STRAIN 32 is a polymyxin free mutant of wild-type isolate P. polymyxa LU17007 and derives from a random mutagenesis approach. The strain was exemplarily chosen to demonstrate spore formation during cultivation, but heterochronicity in spore formation was also proven in wild-type strain P. polymyxa LU17007 and its further mutant successors such as LU54 and LU52, in public Paenibacillus strains such as P. polymyxa DM365 or P. terrae DSM15891, as well as in biocontrol strain Bacillus velenziensis MB1600 (data not shown).

Fermentation conditions to analyze timing of spore formation in Paenibacillus STRAIN 32.

Preculture Conditions

The composition of PX-125 is listed in Table 1. The components of the stock solution were dissolved in distilled water and either sterile filtered or autoclaved at 121° C., 1 bar overpressure for 60 min. The sterile solutions were stored either at room temperature or at 4° C. The antifoam agent was added to the main solution shortly before starting the autoclaving process. After mixing the stock solutions, the pH of the medium was set to 6.5 either with 25% (w/w) ammonia solution or 40% (w/w) phosphoric acid.

TABLE 1
Composition of the complex medium PX-125 with the specification for storage (room temperature (RT)
or 4° C.) and sterilization method (sterile-filtered/autoclaved, s/a) of the stock solution.

| Stock solution | Component | Concentration in the medium [g/l] |
|---|---|---|
| Main solution (RT, a) | Citric acid monohydrate | 3.21 |
| | Dipotassium hydrogen | 1.00 |
| | phosphate | |
| | Ammonium sulfate | 1.07 |
| | Magnesium sulfate | 1.62 |
| | heptahydrate | |
| | Yeast extract | 5.00 |
| | Soy flour | 10.00 |
| | Antifoam | 0.2 |
| Calciumnitrate Tetrahydrate (4° C., s) | Calcium nitrate tetrahydrate | 0.342 |
| Maltose (RT, a) | Maltose monohydrate | 63.00 |
| Vitamin solution (4° C., s) | Thiamin hydrochloride | 0.005 |
| | Nicotinic acid | 0.005 |
| | Riboflavin | 0.0002 |
| | Biotin | 0.00005 |
| | Calcium pantothenate | 0.001 |
| | Pyridoxin hydrochloride | 0.005 |
| | Vitamin B12 | 0.00005 |
| | Lipoic acid | 0.00005 |
| Trace element solution (4° C., s) | Manganese sulfate | 0.013 |
| | monohydrate | |
| | Copper sulfate pentahydrate | 0.0046 |
| | Sodium molybdate dihydrate | 0.0028 |
| | Iron sulfate monohydrate | 0.015 |
| | Citric acid monohydrate | 0.4 |

Preculture cultivation was conducted in 1 L shake flasks with baffles containing 110 ml of culture media PX-125 sealed with breathable silicon plugs. Media was inoculated with 0.6% (v/v) using a cryo culture vial of Paenibacillus STRAIN 32. Cultivation was performed at 33° C., 150 rpm and 25 mm shaking frequency for 24 h.

Main Culture Conditions

Preculture shake flasks were pooled and transferred to the 211 bioreactor containing 121 PX-141 medium (2% inoculation v/v). The recipe of main culture media PX-141 is listed in Table 2.

Main culture medium: PX-141

TABLE 2
Composition of the complex medium PX-141 with the specification for storage (room temperature (RT)
or 4° C.) and sterilization method (sterile-filtered/autoclaved, s/a) of the stock solution.

| Stock solution | Component | Concentration in the medium [g/l] |
|---|---|---|
| Main solution (RT, a) | Citric acid monohydrate | 3.21 |
| | Dipotassium hydrogen | 1.00 |
| | phosphate | |
| | Ammonium sulfate | 1.07 |
| | Magnesium sulfate | 1.62 |
| | heptahydrate | |
| | Yeast extract | 15.00 |
| | Antifoam | 0.2 |
| Calciumnitrate Tetrahydrate (4° C., s) | Calcium nitrate tetrahydrate | 0.342 |
| Maltose (RT, a) | Maltose syrup (50%) | 156.00 |
| Vitamin solution (4° C., s) | Thiamine hydrochloride | 0.005 |
| | Nicotinic acid | 0.005 |
| | Riboflavin | 0.0002 |
| | Biotin | 0.00005 |
| | Calcium pantothenate | 0.001 |
| | Pyridoxine hydrochloride | 0.005 |
| | Vitamin B12 | 0.00005 |
| | Lipoic acid | 0.00005 |
| Trace element solution (4° C., s) | Manganese sulfate | 0.013 |
| | monohydrate | |
| | Copper sulfate pentahydrate | 0.0046 |
| | Sodium molybdate dihydrate | 0.0028 |
| | Iron sulfate monohydrate | 0.015 |
| | Citric acid monohydrate | 0.4 |

Fermentation was carried out at 33° C. for 72 h. The pH was set to 6.5 and adjusted with ammonium hydroxide or phosphoric acid. The dissolved oxygen was set to >20% by regulating stirrer speed (500-1200 rpm) and aeration (5-30 L/min). Fermentation culture samples were taken every 6 h and were stored at 4° C.

Quantification of Spores

Spore counts in fermentation samples were evaluated by phase-contrast microscopy using C-Chip disposable counting chambers (Neubauer/NanoEnTek) according to the manufacturer's manual. For accurate counting, fermentation samples were serially diluted using sterilized 0.9% NaCl solution. Generation of dilution series and counting of spore titer was done in triplicates for each sampling point.

The net production of spores per time interval of the fermentation is shown in FIG. 2.

### Example 2: Outgrowth Timing of Spores Formed at Different Point in Time During Fermentation

Generation of Purified Spore Solutions to Observe Outgrowth Properties

In order to investigate the germination timing of spores formed at different point in time during course of fermentation, purified spore solutions were generated and adjusted to the same spore count/ml according to the following procedure.

At first, vegetative cells were killed by heat treatment of 2 ml culture broth samples harvested at 24, 30, 36, 42, 48, 54, 60, 66 and 72 h cultivation time of the aforementioned fermentation of example 1 at 60° C. for 60 min.

Subsequently, spores were washed by centrifugation with 3,000 g at 4° C. and resuspended with 5 ml sterilized ddH₂O. The washing cycles were performed for at least five times to get rid of cell debris and media residues. Afterwards, the spores were resuspended in 5 ml sterilized ddH₂O and stored overnight at 4° C. The washing cycles were again conducted for at least five times the next day. The purified spore stock was resuspended in 1 ml sterilized ddH₂O and stored at 4° C. Spore purity was assessed by phase-contrast microscopy revealing ≥99% spores while counting ≥200 cells (spores) per microscopic picture section.

The purified spore concentration was then determined by C-Chip counting, as described previously in example 1 and was adjusted with dH20 to same number of spores/samples.

Outgrowth timing of purified spore samples was assessed by monitoring the increase of biomass in microtiter plate cultivations (48-round well-MTP, MTP-R48-BOH, m2p-labs) using a BioLector (m2p-labs) cultivation device.

For this, 10E+6 of purified spores generated in the abovementioned procedure were inoculated into 1.2 ml of PX-131 medium in a 48-round well-plate (MTP-R48-BOH, m2p-labs).

The media recipe of PX-131 used for microtiter plate cultivations is shown in Table 3.

TABLE 3
Composition of the complex medium PX-131 with the specification for storage (room temperature (RT)
or 4° C.) and sterilization method (sterile-filtered/autoclaved, s/a) of the stock solution.

| Stock solution | Component | Concentration in the medium [g/l] |
|---|---|---|
| Main solution (RT, a) | Citric acid monohydrate | 3.21 |
| | Dipotassium hydrogen | 1.00 |
| | phosphate | |
| | Ammonium sulfate | 1.07 |
| | Magnesium sulfate | 1.62 |
| | heptahydrate | |
| | Yeast extract | 10.00 |
| | Antifoam | 0.2 |
| Calciumnitrate Tetrahydrate (4° C., s) | Calcium nitrate tetrahydrate | 0.342 |
| Maltose (RT, a) | Maltose syrup (50%) | 156.00 |
| Vitamin solution (4° C., s) | Thiamine hydrochloride | 0.005 |
| | Nicotinic acid | 0.005 |
| | Riboflavin | 0.0002 |
| | Biotin | 0.00005 |
| | Calcium pantothenate | 0.001 |
| | Pyridoxine hydrochloride | 0.005 |
| | Vitamin B12 | 0.00005 |
| | Lipoic acid | 0.00005 |
| Trace element solution (4° C., s) | Manganese sulfate | 0.013 |
| | monohydrate | |
| | Copper sulfate pentahydrate | 0.0046 |
| | Sodium molybdate dihydrate | 0.0028 |
| | Iron sulfate monohydrate | 0.015 |
| | Citric acid monohydrate | 0.4 |

To reduce evaporation, plates were sealed with gas-permeable sealing foil with evaporation reduction layer (m2p-labs).

Cultivation in 48-well plates was carried out at 900 rpm, 2.5 mm shaking diameter, 33° C. and 85% humidity for at least 72 h. Biomass (A.U.) was measured via scattered light with a wavelength of 620 nm every 15 min.

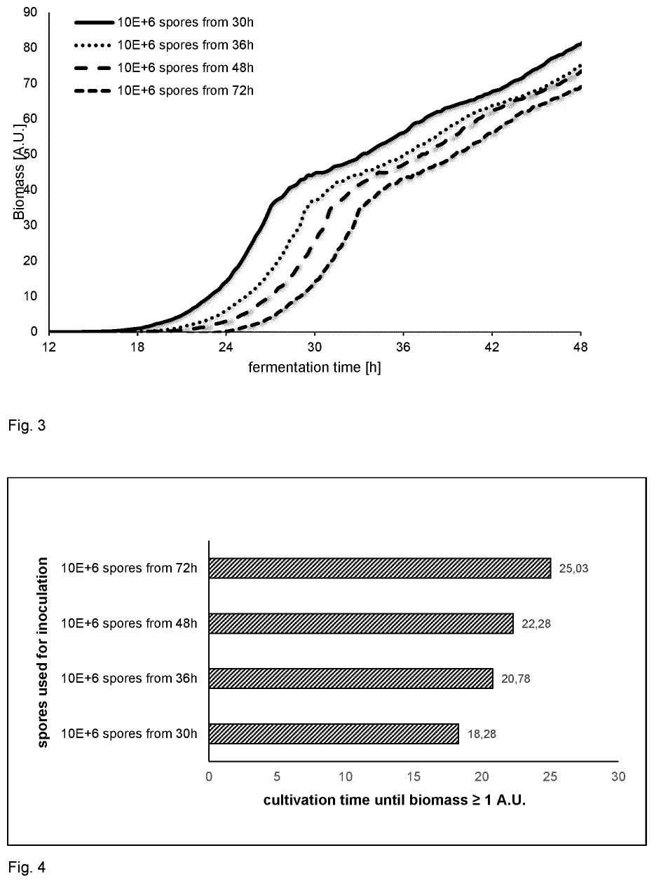

Biomass formation in the MTP scale cultivation of spore samples (10E+6 spores each) harvested after different point in time during course of the fermentation of example 1 are depicted in FIG. 3. The timing of spore outgrowth was defined by reaching a biomass of ≥1 A.U. and is shown in FIG. 4.

### Example 3: Fusaricidin Production of “Early” And“Late” Spore Samples

Production of Fusaricidin was assessed in the fermentation samples of example 2 after 48 h cultivation. For this, 50 μl of culture broth was mixed together with 950 μl acetonitrile-water (1:1) mixture for extraction. The sample was treated for 30 min at 20° C. in an ultrasonic bath. The sample then was centrifuged for 5 min at 14000 rpm and the supernatant filtered into a HPLC vial for measurement. Fusaricidin concentration was determined by HPLC-UV-VIS as listed in Table 5, 5 and Table 6:

TABLE 4
Fluorescence microscopy filter settings

| | | Trans- | | | Exposure |
|---|---|---|---|---|---|
| Channel | Filter | mission | Excitation | Emission | time [sec] |
| Bright field | POL/POL | 32% | — | — | 0.025 |
| Fluorescence | mCherry/ | 100% | 575/25 nm | 632/60 nm | 0.5-3 sec |
| | mCherry | | | | |

TABLE 5
HPLC setting for quantification of Fusaricidin
A, B and C in culture broth samples

| | |
|---|---|
| Column: | Aqua C18, 250*4.6 mm (Phenomenex) |
| Pre-column: | C18 Aqua |
| Temperature: | 40° C. |
| Flowrate: | 1.00 mL/min |
| Injection volume: | 2.0 μL |
| Detection: | UV 200 nm |
| Maximal pressure: | 400 bar |
| Stop time | 20 min |
| Eluent A: | H₂O with 0.1% H3PO4 |
| Eluent B: | acetonitrile |

TABLE 6
Solvent gradient for HPLC based quantification of
Fusaricidin A, B and C in culture broth samples

| Time [min] | A [%] | B [%] | Flow [ml/min] |
|---|---|---|---|
| 0.0 | 70.0 | 30.0 | 1.00 |
| 6.0 | 60.0 | 40.0 | 1.00 |
| 12.0 | 0.00 | 100.0 | 1.00 |
| 16.0 | 0.00 | 100.0 | 1.00 |
| 16.10 | 70.0 | 30 | 1.00 |

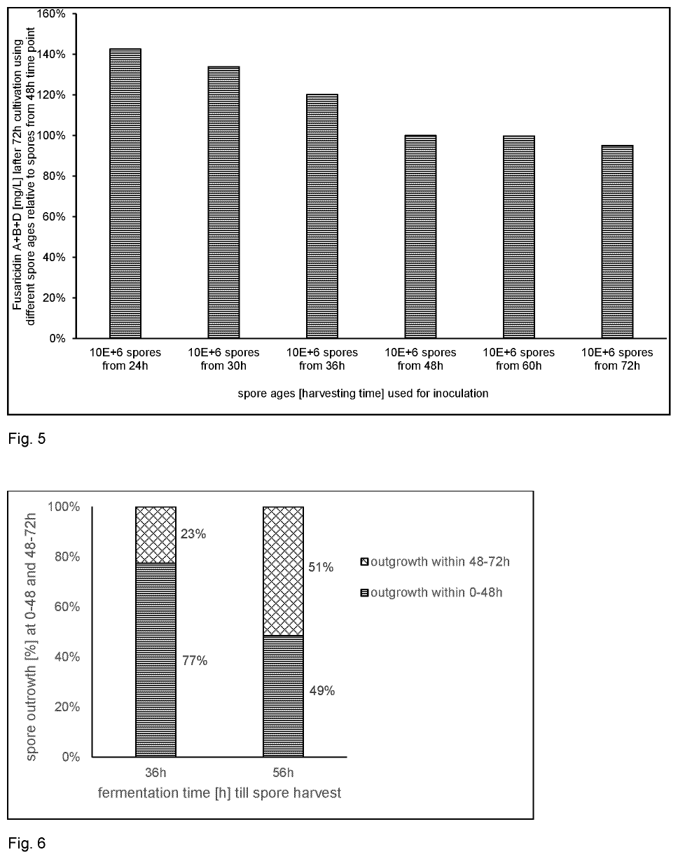

The production of Fusaricidin A, B and D in the cultivation of example 2 is shown in FIG. 5.

### Example 4: Ratio of Early and Late Spores in Pilot Scale Fermentations at Different Point in Time

Fermentation conditions to collect spores from different point in time

Preculture Conditions

Preculture shake flasks for Paenibacillus STRAIN 32 were handled as described in example 1 using PX-125 medium. Only maltose level was reduced to 30 g/L. After 21.5 h cultivation time, shake flask preculture was used for inoculation (1.5% v/v) of a 21 l bioreactor filled with 12 l PX-172 medium listed in Table 7.

TABLE 7
Media recipe of PX-172 main culture medium

| Stock solution | Component | Concentration in the medium [g/l] |
|---|---|---|
| Main solution (RT, a) | Citric acid monohydrate | 3.21 |
| | Dipotassium hydrogen | 1.00 |
| | phosphate | |
| | Ammonium sulfate | 1.07 |
| | Magnesium sulfate | 1.62 |
| | heptahydrate | |
| | Soy flour | 13.00 |
| | Antifoam | 0.2 |
| Calciumnitrate Tetrahydrate (4° C., s) | Calcium nitrate tetrahydrate | 0.342 |
| Maltose (RT, a) | Maltose syrup (50%) | 156.00 |
| Vitamin solution (4° C., s) | Nicotinic acid | 0.005 |
| | Biotin | 0.00005 |
| Trace element solution (4° C., s) | Manganese sulfate | 0.013 |
| | monohydrate | |
| | Copper sulfate pentahydrate | 0.0046 |
| | Sodium molybdate dihydrate | 0.0028 |
| | Iron sulfate monohydrate | 0.015 |
| | Citric acid monohydrate | 0.4 |
| Amino acids | DL-Methionine | 0.4 |

Fermentation was carried out as described in example 1 at 33° C. for 18 h and was then transferred in a 300 L main culture fermenter containing 1801 again PX-172 medium. Main fermentation was carried out at 33° C. for 72 h. The pH was set to 6.5 and adjusted with ammonium hydroxide or phosphoric acid. The dissolved oxygen was set to >20% by regulating stirrer speed (300-600 rpm) and aeration (2.5-12 m³/h). Fermentation culture samples were taken every 6 h and were stored at 4° C.

To identify the ratio of fast and slow germinating spores of Paenibacillus polymyxa STRAIN 32 at different point in time of fermentation, culture broth samples were taken from the above-mentioned fermentation after 36 h and 56 h cultivation time.

For this, 100 μL of culture broth was diluted with 900 μL of a sterile 0.9% NaCl 0.9% NaCl+0.1 g/L Tween 80 solution. Using 2 ml tubes, the mixture was further diluted in decadic steps using the same diluent up to a final dilution level of 10E-9.

Then, each culture dilution step was heated in a thermocycler at 60° C. for 30 min to kill vegetative cells. 100 μl of each approach was plated on an ISP2 agar plate and subsequently cultivated for 72 h at 33° C. The recipe of ISP2 agar is shown in Table 8.

TABLE 8
Composition of the ISP2 agar medium. All components were mixed
together, autoclaved and stored at room temperature.

| Component | Concentration in the medium [g/l] |
|---|---|
| Yeast extract | 4 |
| Dextrose | 4 |
| Malt extract | 10 |
| Agar | 15 |
| Water | Add 1L |

Colony forming units (CFU) on agar plates were determined after 48 h and 72 h of cultivation by counting. Ratio of CFUs found after both assessment time points are shown in FIG. 6.

### Example 5: Dipicolinic Acid (DPA) Levels of Early and Late Spores of Paenibacillus in Fermentation Samples

DPA extraction from spores was conducted according to the following procedure:
1. Spore pellet generation: centrifuge 10 ml of fermentation broth at 18,000 g for 10 min
2. Carefully discard supernatant
3. Add 10 ml sterile dH20 and dissolve pellet by shaking and inverting via pipette to wash the spore pellet
4. Centrifuge washed spore solution at 18,000 g for 10 min, discard supernatant
5. Repeat washing steps 3-4
6. Resuspend pellet in 5 ml dH20 and dissolve pellet by shaking and inverting via pipette
7. Transfer whole approach into pressure-resistant 30 ml glass injection-vessels and seal with butyl rubber stops. Seal with aluminum crimps
8. Autoclave sample at 121° C. for 60 min
9. After cooling, open glass vessel and transfer 2 ml into a 2 ml micro centrifuge tube. Perform centrifugation at 18,000 g for 10 min
10. Transfer and filter supernatant into a HPLC analyses vial DPA level was quantified by HPLC UV-VIS according to the parameters listed in Table 9 and Table 10.

TABLE 9
HPLC setting for DPA quantification in culture broth samples

| | |
|---|---|
| Column | Aqua C18, 250*4.6 mm (Phenomenex) |
| Precolumn | Aqua C18 |
| Temperature | 40° C. |
| Flow rate | 1.00 ml/min |
| Injection volume | 5.0 μl |
| Detection | UV 222 nm |
| Run time | 17.0 min |
| Max. pressure | 250 bar |
| Eluent A | 10 mM KH2PO4, pH 2.5 |
| Eluent B | Acetonitrile |

TABLE 10
Solvent gradient for HPLC based quantification
of DPA in culture broth samples

| Time [min] | A [%] | B [%] | Flow [ml/min] |
|---|---|---|---|
| 0.0 | 93.0 | 7.0 | 1.0 |
| 10.0 | 93.0 | 7.0 | 1.0 |
| 12.0 | 50.0 | 50.0 | 1.0 |

Calibration curve was set up using 0.1, 0.5 and 1 mM 99% dipicolinic acid. Dipicolinic acid was detected at 5.7 min retention time.

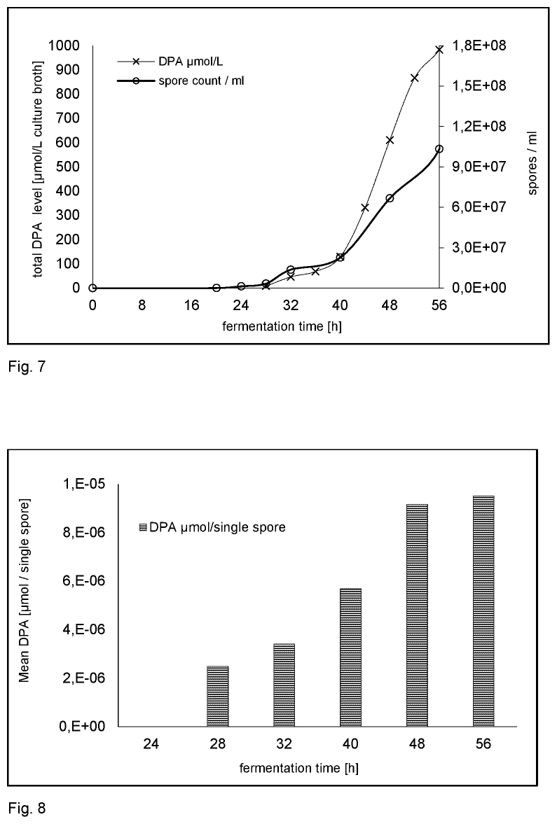

Using this method, total DPA level/ml fermentation broth was analyzed in culture broth samples of the fermentation carried out in example 3 taken over the course of the cultivation. In parallel, viable spore titer was assessed by the dilution and plate counting as described in example 4. Results are shown in FIG. 7.

Based on this, ratio of DPA per single spore was calculated by using the formula

$$D P A μ mol / single = \frac{DPA μmol / ml fermentation broth}{spore count / ml fermentation broth}$$

and the resulting ratios for different time points are shown in FIG. 8.

### Example 9: Heterochronic Outgrowth of Spores from Clostridia

To assess timing of spore outgrowth of other spore forming bacteria than Bacillus and Paenibacillus, two strains from the genus Clostridium, namely C. tetanomorphum DSM528 and C. tyrobutyricum DSM1460, were exemplary chosen for further characterization. Both strains were cultivated for 5 days on RCM agar at 28° C. under anaerobic conditions. Recipe of RCM agar is shown in Table 11. After this, 5 individual colonies were picked and transferred in liquid culture vials containing 6 ml of TSB broth. Recipe of TSB broth media is shown in Table 12. All steps were performed in biological triplicates under anaerobic conditions using an anaerobic clove box. Liquid cultures of C. tetanomorphum DSM528 and C. tyrobutyricum DSM1460 were cultured for 7 days at 28° C. well into sporulation. To analyze timing of spore outgrowth, 1 ml of each liquid culture was heated at 60° C. for 30 min to kill remaining vegetative cells. Then, 100 μl of each approach was plated on TSB agar (Table 12). Agar cultures were grown for 96 h at 28° C. under anaerobic conditions. Colony forming units (CFU) were counted after 48 h and 96 h cultivation. Ratios of CFU found after 48 h and 96 h cultivation time relating to the total CFU counts from 96 h are shown in FIG. 9.

TABLE 11
Composition of RCM agar for growth of Clostridia, pH: 6.8 ± 0.2

| Component | Concentration in the medium [g/l] |
|---|---|
| Beef extract | 10 |
| Casein enzymatic hydrolysate | 10 |
| L-cysteine hydrochloride | 0.5 |
| Dextrose | 5 |
| Sodium acetate | 3 |
| Sodium chloride | 5 |
| Starch soluble | 1 |
| Yeast extract | 3 |

TABLE 12
Composition of TSB broth and agar for
growth of Clostridia, pH: 7.3 ± 0.2

| Component | Concentration in the medium [g/l] |
|---|---|
| Casein peptone (pancreatic) | 17 |
| Soy peptone | 3 |
| Dextrose | 2.5 |
| Sodium chloride | 5 |
| Dipotassium phosphate | 2 |
| (Agar) | (15) |

1. A spore composition comprising purified spores of a prokaryotic microorganism, wherein
a) said spores form colonies when plated on a medium suitable for colony formation, and wherein of all such colonies formed within 72 h for aerobic cultures and 96 h for anaerobic cultures after plating at least 40% are formed within 48 h, and/or
b) at least 40% of spores are obtainable or obtained from a fermentation harvested during a first spore formation phase and/or
c) the mean content of dipicolinic acid per spore is at most 80% of the mean content of dipicolinic acid of spores fermented in a suitable medium until plateau phase.

2. The composition according to claim 1, wherein the microorganism is selected from the group consisting of the taxonomic rank of phylum Firmicutes, class Bacilli, Clostridia and Negativicutes.

3. The composition according to claim 1, wherein the composition
a) comprises viable cells and spores in a ratio of at most 4.1, and/or
b) comprises, in addition to said spores, at least one pest control agent,
and/or
c) comprises at least one fusaricidin, paeniserine or paeniprolixine and/or
d) comprises at least one auxiliary selected from the group consisting of stabilisers, extenders, solvents, surfactants, spontaneity promoters, solid carriers, liquid carriers, emulsifiers, dispersants, film forming agents, frost protectants, thickeners, plant growth regulators, inorganic phosphates, fertilizers, adjuvants, spore germinant, fatty acids and fibril, microfibril and nanofibril structuring agents.

4. The composition according to claim 1, wherein the composition is a plant pest control composition and/or prevents, limits or reduces a phytopathogenic fungal or bacterial disease and/or improves or promotes the health of a plant and/or increases or promotes yield of plants when applied to such plant, a part or propagation material thereof or to the substrate where the plants are to grow.

5. The composition according to claim 1, wherein the composition comprises at least 10\hat{ }4 cfu/ml of said spores.

6. The composition according to claim 1, wherein at least a fraction of the spores comprises on their surface a protein comprising a payload domain, said protein also comprising a targeting domain for delivery of the payload domain to the surface of said spores.

7. A plant protection product, comprising a plant cultivation substrate coated or infused with the composition according to claim 1.

8. A plant, plant part or plant propagation material, wherein the material comprises, on its surface or infused therein, the composition according to claim 1.

9. A plantation comprising the plant, plant part or plant propagation material according to claim 8.

10. A cleaning product comprising the composition according to claim 1.

11. A food, feed or cosmetic product comprising the composition according to claim 1.

12. A building product comprising the composition according to claim 1.

13. A method of producing a composition comprising spores of a prokaryotic microorganism, the method comprising the steps of
1) fermenting the microorganism in a medium conductive to sporulation, and
2) purifying the spores to obtain the composition,
wherein
a) purification is performed latest when 85% of the maximum spore concentration obtainable in the fermentation step 1) is reached and/or
b) purification is performed such that said purified spores form colonies when plated on a medium suitable for colony formation, and wherein of all such colonies formed within 72 h for aerobic cultures and 96 h for anaerobic cultures after plating at least 40% are formed within 48 h, and/or
c) purification is performed such that said purified at least 40% of spores are obtainable or obtained from a fermentation harvested during a first spore formation phase and/or
d) purification is performed when the mean content of dipicolinic acid per spore is at most 80% of the mean content of dipicolinic acid of spores produced when reaching maximum spore concentration in the fermentation step 1).

14. The method according to claim 13, wherein the microorganism is selected from the group consisting of taxonomic rank of of phylum Firmicutes, class Bacilli, Clostridia and Negativicutes.

15. The method according to claim 13, wherein
a) the purification step 2)
comprises a step of desiccation, lyophilization, homogenization, extraction, filtration, centrifugation, sedimentation, or concentration of spores, and/or
comprises adjusting the water content of the composition to
a) for dry, powder or granular compositions: 1-10% by weight of the composition,
b) for liquid or pasty compositions 10-98% by weight of the composition, and/or
comprises adjusting the carbon source content of the composition to at most 50% by weight of the composition compared to its content at the time of spore harvest,
results in a suppression or reduction of spore germination in the composition, and/or
b) the method further comprises addition of at least one pest control agent,
c) the method further comprises addition of at least one fusaricidin, paeniserine or paeniprolixine, wherein the one or more fusaricidins comprise any of fusaricidin A, B or D, and/or surfactin and/or iturin, and/or
d) the method further comprises addition of at least one auxiliary selected from the group consisting of stabilisers, extenders, solvents, surfactants, spontaneity promoters, solid carriers, liquid carriers, emulsifiers, dispersants, film forming agents, frost protectants, thickeners, plant growth regulators, inorganic phosphates, fertilizers, adjuvants, spore germinant, fatty acids and fibril, microfibril and nanofibril structuring agents.

16. A method for fermentation, comprising the step of inoculating a fermenter comprising a suitable fermentation medium with the composition according to claim 1.

17. A method for controlling, in a fermentation of spore-forming prokaryotic microorganisms, the duration of a lag phase and/or the time until reaching the end of log phase, comprising inoculating a suitable fermentation medium with the composition according to claim 1 and fermenting the inoculated medium, wherein for shorter duration of the lag phase and/or faster end of log phase a composition is used having a higher percentage of spores harvested in a first spore formation phase, and for longer duration of lag phase or later end of log phase a composition is used having a higher percentage of spores harvested in a second spore formation phase.

18. A computer-implemented method for providing an inoculant sample for fermentation, comprising the steps of
i) obtaining a target duration of the lag phase and/or end of log phase,
ii) calculating the required percentage of spores harvested during the first spore formation phase and/or the second spore formation phase, and
iii) performing a reaction based on the calculation in step 2 selected from the group consisting of:
(1) emission of an identifier of an inoculant sample of a working cell bank sample collection best fitting to the calculated ratio,
(2) retrieval of an inoculant sample of a working cell bank sample collection best fitting to the calculated ratio,
(3) dosing of an inoculant sample of a working cell bank sample collection best fitting to the calculated ratio to the fermenter, and
(4) mixing of a new working cell bank sample by adjusting the proportion of early and late spore communities by drawing from an early spore community enriched and from a late spore community enriched stock, respectively, and optionally dosing said mixture to the fermenter.

19. A method of promoting spore germination and/or vegetative growth of a spore-forming prokaryotic microorganism, comprising providing spores harvested during a first spore formation phase in the method according to claim 13.

20. A method of using the composition according to claim 1, the method comprising using the composition:
a) for inoculating a fermentation, or
b) for pest control and/or for preventing, delaying, limiting or reducing the intensity of a phytopathogenic fungal or bacterial disease and/or for improving the health of a plant and/or for increasing yield of plants and/of for preventing, delaying, limiting or reducing the emission of phytopathogenic fungal or bacterial material from a plant cultivation area, or
c) for the preparation of a plant protection product, or
d) for the preparation of a probiotic food, feed or cosmetic formulation, or
e) for the preparation of a cleaning product, or
e) for the preparation of a concrete.

21. A method of protecting a plant or part thereof in need of protection from pest damage, comprising contacting the pest, plant, a part or propagation material thereof or to the substrate where the plants are to grow with an effective amount of a composition according to claim 1.

22. A method of delivering a protein payload to a plant, plant art, seed or growth substrate, comprising applying the composition according to claim 1 to the plant, plant part, seed or substrate, wherein the spores are those of a microorganism expressing a protein comprising a payload domain and a targeting domain for delivery of the payload domain to the surface of said spores.

23. A method of using the composition according to claim 20, the method comprising using the composition wherein
i) the fungal disease is selected from the group consisting of white blister, downy mildews, powdery mildews, clubroot, sclerotinia rot, fusarium wilts and rots, botrytis rots, anthracnose, rhizoctonia rots, damping-off, cavity spot, tuber diseases, rusts, black root rot, target spot, aphanomyces root rot, ascochyta collar rot, gummy stem blight, alternaria leaf spot, black leg, ring spot, late blight, cercospora, leaf blight, septoria spot, leaf blight, and a combination thereof, and/or
ii) the fungal disease is caused or aggravated by a microorganism selected from the group consisting of the taxonomic ranks:
class Sordariomycetes;
class Sordariomycetes order Glomerellalese;
class Leotinomycetes;
class Dothideomycetes;
class Dothideomycetes of order Pleosporales;
class Dothideomycetes of order Botryosphaeriales;
class Dothideomycetes of order Capnodiales;
class Agraricomycetes;
class Pucciniomycetes;
class Ustilaginomycetes;
class Oomycota;
class Oomycota of order Pythiales; and
class Oomycota of order Peronosporales.
